(19) United States
(12) Patent Application Publication
(10) Pub. No.: US 2004/0267567 A1
(43) Pub. Date: Dec. 30, 2004

(54) Hospitality management system and methods

(76) Inventors: Javier Barrera, Cuajimalpa (MX); Jose Carlos Azcarraga, Cuajimalpa (MX)

Correspondence Address: WHITE & CASE LLP, PATENT DEPARTMENT, 1155 AVENUE OF THE AMERICAS, NEW YORK, NY, 10036, US

(21) Appl. No.: 10/765,245

(22) Filed: Jan. 26, 2004

Related U.S. Application Data
(60) Provisional application No. 60/442,198, filed on Jan. 24, 2003.

(51) Int. Cl. G06F 17/60; G06G 7/00; G06F 17/00

(52) U.S. Cl. 705/2; 705/28; 705/400

(57) ABSTRACT

The present invention is directed to methods and systems for managing a hospitality organization having geographically distributed business entities each providing one or more respective, revenue generating facilities such as hotel rooms, banquet facilities and the like. Arrangements with respect to use of the facilities provided by the business entities are made via one or more of a plurality of channels. In an embodiment of the invention, a method includes the steps of maintaining a centralized inventory system for the business entities and the respective facilities associated with the business entities, receiving via at least one of the plurality of channels a request for a pricing proposal associated with at least one of the facilities of at least one of the business entities, and, in response to the request for the pricing proposal associated with the at least one facility, generating a quote based on data residing in the centralized inventory system and transmitting the quote, via at least one of the plurality of channels, in response to the request for the pricing proposal.

This application claims the benefit, under 35 U.S.C. § 119(e), of provisional patent application No. 60/442,198, filed on Jan. 24, 2003, the contents of which are incorporated by reference herein in their entirety.

### FIELD OF INVENTION

The present invention relates in general, to hospitality management, in particular, hospitality management systems and methods.

### BACKGROUND OF INVENTION

Hospitality organizations, particularly those having numerous geographically distributed business entities and facilities, are continually confronted by the challenge of pricing and booking the facilities of these businesses in order to maximize the organization's profit. Doing so, in turn, involves reckoning with the myriad distribution channels via which price enquiries, reservations, and bookings are done, as well with as the distributed, heterogeneous natures of the business entities, their management and pricing practices, and their information systems. The distributed, heavily customized nature of conventional hospitality organizations and their information infrastructures are expensive to maintain, difficult to integrate with and/or migrate across existing or emerging technology platforms or solutions, and require extensive interfacing and translation between the different data structures and repositories of the existing customized solutions of each of the distributed business entities.

At the same time, these organizations are limited in their ability to access or leverage the valuable business transaction information residing locally at the distributed business entities. These limitations result from the distributed nature of the organization's information management, the significant hardware investment that is required to provide information access, and the overhead associated with bottlenecks that occur between custom interfaces of a organization's distributed business entities and facilities. As a hospitality organization expands and acquires other business entities, technology solutions tend to be patched onto the legacy systems, further adding to the complexity and disjointedness of the architecture of the organizations information systems. Business data, the information that can be gleaned from it, and decision making regarding such matters as pricing of facilities, tends to remain localized.

FIG. 1 shows what is believed to be a typical example of an architecture of a conventional hospitality management system, of the sort typically used by a hotel organization having a number of geographically distributed business entities. System 100 encompasses a plurality of geographically distributed business entities 102 of a hospitality organization, such as entity 106, 108, 112, and 114, whereby each entity includes a local repository or database 104. Each local data base 104 may hold booking data, data on customers and groups, and other administrative and marketing data that is pertinent to the day-to-day operation of the respective business entity. For example, business entities 106 and 108 are located in geographical location 110, which may be, for example, a city, region, country, or even continent. Similarly, business entities 112 and 114 are located in a different geographical location 116.

As illustrated in FIG. 1, each business entity 102 is limited to accessing local data associated with their local databases 104, and does not in general have access to data associated with the other business entities or other sources.

Business entity 106 may possess important data related to a particular customer or group stored in its database 104. This customer or group may, at a later date decide to make a room reservation at another business entity within the hospitality organization, such as entity 112. Business entity 112 may not, in general, have access to the data related to the customer or group from business entity 106. The customer information at entity 106 may indicate that the customer (or group) is a regular customer with certain preferences, which, if taken into consideration during the booking process, might be considered in setting a profitable room rate at entity 112. As previously mentioned, entity 112 may not be able to access information from the local database of entity 106 or to do so on a basis that would enable use of the information in booking facilities. Moreover, even if there is a communication infrastructure between entities 106 and 112, there is no guarantee that the data is accessible in a short enough time period for it to be utilized by entity 112. In other words, the information is not available in real-time.

As shown in FIG. 1, system interconnection block 120 provides communication means between customers 122 and business entities 102. The customers 122 may be individuals 124 wishing to make a reservation, a company 126, a group 128, or a wholesaler 130. Each of the customers 122 can initiate the booking process through various distribution channels 132, such as a walk-In 134, through a global distribution system (GDS) 136, the hospitality organization's call reservation center 138, online merchants 140 (e.g., Travelocity™, Expedia™, etc.), or other channels.

If an individual 124 intends to make a reservation or room rate inquiry, they may do so over a number of channels 132. For example, they may contact the call reservation center 138 for a particular room on a particular date. From the call center, data on the individual and the request is sent over communications channel 142, interface (I5) 144, and interface (I2) 146 to business entity 106, where the request is processed based on the received data, and other required information data stored in database 104. Prior to the request data being processed at entity 106, customized interface (I2) 146 may need to translate or format the request data (i.e., from call center) so that is compatible with the information technology (IT) system incorporated at business entity 106. Once the request has been processed at entity 106, room rate data is sent back to the reservation center 138 via communications channel 142, interface (I2) 146, and interface (I5) 144. As with the request data, before the room rate data is updated at call reservation center 138, customized interface 144 must translate or format the request data (i.e., from call reservation center 138) to a format that is compatible with the IT system operating at the call reservation center 138. This interfacing may be required for each of the customers 122 that is communicating with a particular one of the business entities 102 via one of distribution channels 132.

As illustrated in FIG. 1, the different distribution channels 132 and business entities 102 typically may need to communicate via customized interfaces, such as interfaces 144, 146, 148, 150, 152, 154, etc. For simplicity, only a limited number of business entities, distribution channels, and interfaces have been shown. For a hospitality organization having a large number of business entities, many interfaces may be required. Also, when a booking or reservation enquiry has been made via, for example, the GDS 136 or call reservation center (CRS) 138, the database (not shown) for these systems (i.e., GDS and CRS) may require manual updating, with no automatic synchronization.

Consequently, information sharing and coordination between business entities is suboptimal and prices for room reservations or other facilities, are often set at the discretion of each entity's management function, rather than benefiting from any centralized, coordinated mechanisms for utilizing intelligence gathered across the hospitality organization as a whole. This data, if any, across the hospitality organization's business entities, severely restricts the revenue generation of hospitality organizations.

### SUMMARY OF THE INVENTION

The long felt, but unmet, needs described above are addressed, at least in part, by various aspects of the systems and methods according to the present invention. The hospitality management system, in accordance with present invention, among other advantages, provides a centralized system for storing, managing, and processing data associated with business transactions that occur between customers, channels and business entities within a hospitality organization. Access to such centralized data, enables the hospitality management system to maximize or otherwise improve its revenue management practices, provides a flexible means for integrating other properties into the organization's management system infrastructure, and enables generation of a uniform pricing strategy over the various distribution channels that are used to initiate and close business transactions with the business entities.

In particular, the system and methods of the present invention involve a method for managing a hospitality organization having geographically distributed business entities providing one or more respective facilities, wherein arrangements with respect to use of the facilities provided by the business entities are made via one or more of a plurality of channels. The method comprises the steps of: maintaining a centralized inventory system for the business entities and the respective facilities associated with the business entities; receiving via at least one of the plurality of channels a request for a pricing proposal associated with at least one of the facilities of at least one of the business entities; in response to the request for the pricing proposal associated with the at least one facility, generating a quote based on data residing in the centralized inventory system; and transmitting the quote, via at least one of the plurality of channels, in response to the request for the pricing proposal.

Another aspect of the present invention includes a hospitality management system for providing pricing proposals associated with facilities of geographically distributed business entities of a hospitality organization, the hospitality management system comprising: a centralized inventory system comprising a data storage system for storage and retrieval of data associated with booking the facilities of any of the business entities; and a central interface in communication with the centralized inventory system and the business entities and accessible by customer entities for booking at least one of the facilities of at least one of the business entities, the centralized inventory system adapted for generating quotes based on data stored in the data storage system and associated with the facilities of the business entities. The pricing proposals may, according to an aspect of the present invention, be real-time proposals or quotes.

Yet another aspect of the present invention involves a method for operating a central inventory system for a hospitality organization having a plurality of geographically distributed business entities. The method comprises the steps of: maintaining a database associated with the central inventory system, the database comprising centrally-generated price and availability data relating to facilities of the plurality of business entities; receiving a booking request for at least one facility of the plurality of business entities; based on the booking request, retrieving from the database data relating to the facility; processing the retrieved data to generate a quote for the facility; transmitting the quote in response to the booking request; receiving a signal reflecting acceptance of the quote; and updating the database based on receipt of the signal reflecting acceptance of the quote.

Also, another aspect of the present invention includes a centralized system for managing pricing and booking of facilities of geographically distributed business entities of a hospitality organization, the centralized system comprising: a centralized inventory system for maintaining a single repository of data associated with pricing and booking of the facilities; an application server in communication with the centralized inventory system over a network, the application server being accessible over the network by the centralized inventory system for booking the facility; and a central interface in communication with the centralized inventory system, the application server and at least one external system, for supporting communications between the centralized inventory system, the application server, and the at least one external system.

Also, yet another aspect of the present invention involves a method for managing one of a plurality of business entities of a hospitality organization. The method comprises the steps of receiving over a network, from an inventory system centralized with respect to the plurality of business entities, data associated with booking of facilities of the business entity and assigning resources of the business entity based on the booking data received from the centralized system.

### BRIEF DESCRIPTION OF THE DRAWINGS

FIG. 1 illustrates an overview of a hospitality management system, according to existing prior art system.

FIG. 2 illustrates an overview of a hospitality management system used by a hospitality organization in an embodiment of an aspect of the present invention.

FIG. 3 illustrates the system architecture of the hospitality management system in an embodiment of an aspect of the present invention.

FIG. 4 illustrates the interaction of a centralized inventory system with other systems and entities within the hospitality management system in an embodiment of an aspect of the present invention.

FIG. 5 illustrates the flow of data between sales agents of the hospitality organization and the hospitality management system in an embodiment of an aspect of the present invention.

FIG. 6 illustrates communication of data within various components of the hospitality management system in an embodiment of an aspect of the present invention.

FIG. 7 illustrates information processing by the hospitality management system in an embodiment of an aspect of the present invention.

FIG. 8A illustrates a flow diagram of methods associated with the hospitality management system, in an embodiment of an aspect of the present invention.

FIG. 8B illustrates further steps of the methods illustrated in FIG. 8A in an embodiment of the present invention.

FIG. 9A illustrates an alternative flow diagram of methods associated with the hospitality management system in an embodiment of an aspect of the present invention.

FIG. 9B shows subsequent steps of the methods of FIG. 9A in an embodiment of the present invention.

FIG. 10A illustrates a flow diagram for the methods associated with the reservations and the centralized commission payment system of the hospitality management system in an embodiment of an aspect of the present information.

FIG. 10B shows subsequent steps of the methods of FIG. 10A in an embodiment of the present invention.

FIG. 10C shows further steps of the methods of FIG. 10B in an embodiment of the present invention.

FIG. 11 shows an example of a user interface for making reservations for rooms or other facilities of the hospitality organization through a call reservation center in an embodiment of an aspect of the present invention.

FIG. 12 shows an example of a user interface for indicating rates and availability information associated with reservations made through a call reservation center in an embodiment of an aspect of the present invention.

FIG. 13 shows a rate detail user interface shown within the user interface of FIG. 12 in an embodiment of an aspect of the present invention.

FIG. 14 shows an example of a user interface associated with an Internet Home Page of the hospitality organization in an embodiment of an aspect of the present invention.

FIG. 15 shows a user interface used by a sales force automation (SFA) system associated with the hospitality organization for making group reservations according to an embodiment of an aspect of the present invention.

FIG. 16 shows a user interface used by a revenue manager for making manual entries into the hospitality management system in an embodiment of an aspect of the present invention.

### DETAILED DESCRIPTION

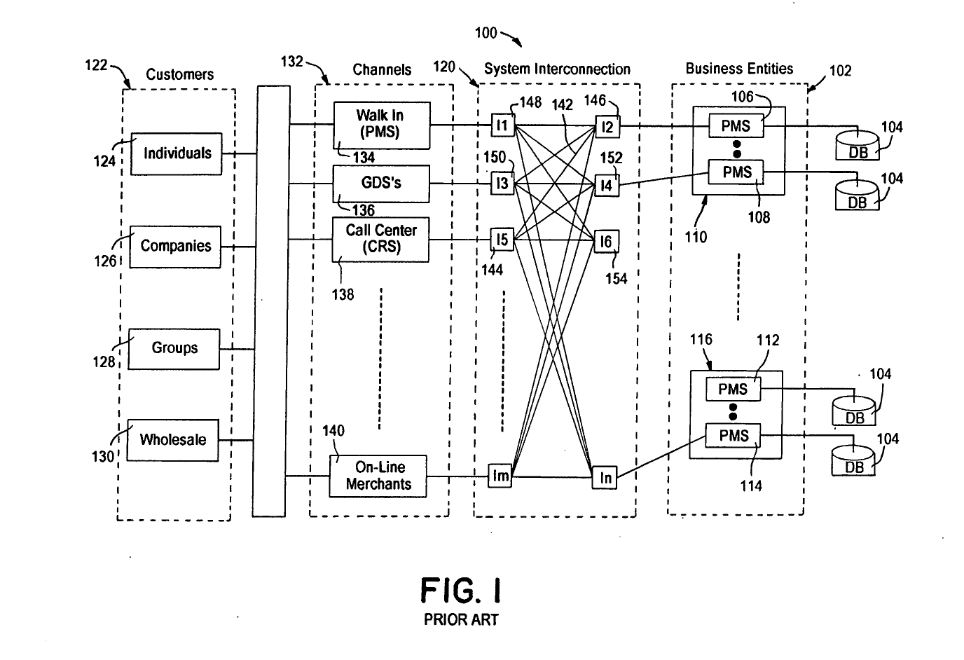

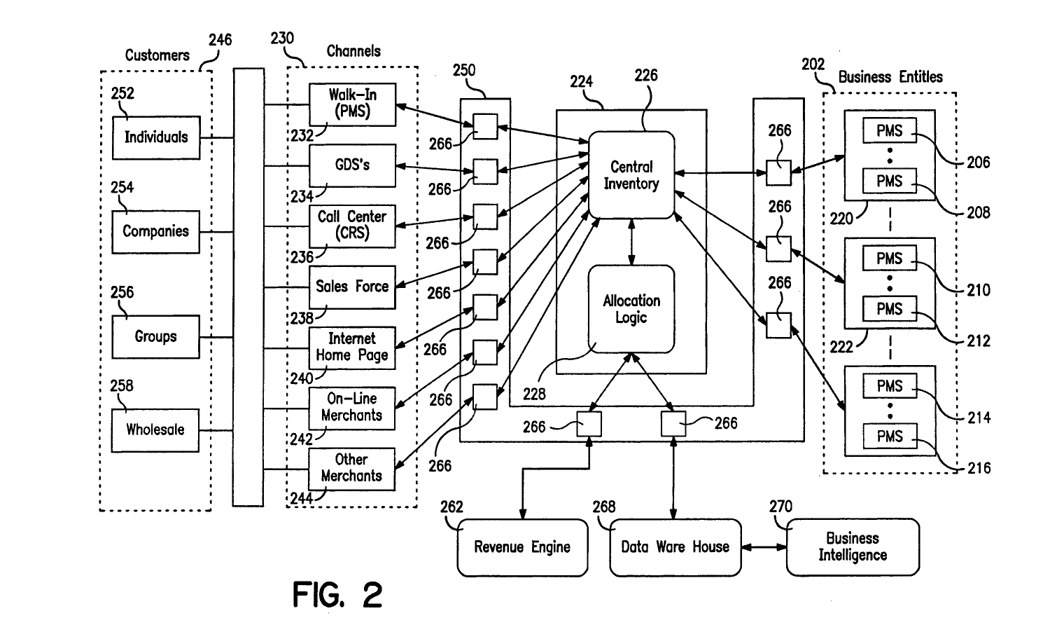

FIG. 2 illustrates an architectural overview of a hospitality management system 200 in an embodiment of an aspect of the present invention. In this embodiment, but without limitation, the hospitality management system is adopted for managing a hotel organization, but could also be used in running a cruise line that operates a number of ships, a vacation club, a time-share condominium organization or any other hospitality organization. As described in relation to FIG. 1, system 200 comprises a plurality of geographically distributed business entities 202, such as entity 206, 208, 212, 214, 216, and 218. As previously discussed, business entities such as, 206 and 208 are located in a particular geographical location 220, while business entities 210 and 212 are located in different geographical location 222. Business entities 210 and 212 may, for example, be in different regions, states, countries, or continents.

The number of business entities 202 may vary greatly. The several business entities (e.g., 206, 208, 210, 212, 214, and 216) entities shown in FIG. 2 are for purposes of illustration and not limitation.

All data regarding the business entities and the facilities, amenities, and services they provide, reside on a centralized inventory system 224. According to an aspect of the present invention, transaction, with a business entity 202 of the hospitality organization, such as a hotel room reservation, must be conducted through centralized inventory system 224.

Centralized inventory system 224 comprises a central repository 226 for storing data associated with hotel availability, rates (or quotes), and bookings (i.e., reservations), whereby the repository may include any system for storing and retrieving data, including without limitation a database. It also comprises an allocation logic module 228, which uses revenue management business rules of the hospitality organization to calculate and optimize rates for reservation proposals received through the various distribution channels. The allocation logic module 228 comprises software that is associated with the central repository 226. Rates for facilities are generated based on revenue management rules, such as, stay patterns, oversell limits and rate hurdles. Therefore, the generation of rates and availabilities are all processed within the centralized inventory system 224, without the burden of interfacing different booking requests or reservation requests through individual business entities and their respective local databases, e.g., entities 106, 108, 112, or 114 shown in FIG. 1.

Various distribution channels 230, including without limitation, walk-ins 232, global distribution systems 234, call reservation systems 236, an organization sales force 238, Internet home page 240, on-line merchants 242, and other merchants 244, each provide customers 246 with access to the centralized inventory system 224 of the hospitality management system 200 via a central interface 250. Customers 246, such as individuals 252, companies 254, groups 256, or wholesalers 258 can each request a reservation or booking through one of the distribution channels 230. Other distribution channels (not shown) can also be dynamically added to the existing channels 230. The customers 246 and channels 230 can collectively be thought of as customer entities, which are customers or others acting or serving on their behalf or in connection with their arrangements, including travel agents, sales staff of the hospitality organization, sales representatives of the business entities, and the like.

A revenue management engine 262, which may be a yield management tool, may be invoked to improve revenue performance based on stay patterns and historical booking data that is stored in repository 226. All historical data that is stored in both the repository 226 and revenue management engine's repository (not shown) is updated, preferably in real-time, as it is created (e.g., through booking data), which enables the revenue management engine 262 to maximize revenues based on the centralized, up-to-date, and efficiently maintained data repository 226. The yield management tool used as part of the revenue management engine 262 may be TopLine PROFI™ (TLP) ENTERPRISE Central Yield Management System licensed by Micros-Fidelio, headquartered in San Jose, Calif., or other suitable tool.

The revenue management engine is most effective when the occupancy levels of the business entities 202 are high. Under these circumstances, forecasting transient future demands using the revenue management engine 262, enables business entities 202 to restrict the availability of a certain portion or percentage of their rooms and facilities (e.g., conference resources) for transient sales (i.e., walk-in sales). Therefore, by accounting for transient bookings as opposed to other less profitable channels (e.g., GDS 234), revenues can be increased, in an embodiment of the invention and without limitation, by up to 5% compared to traditional hospitality management systems. Traditional hospitality management systems typically may not include revenue management business rules and may use separate repositories for each distribution channel.

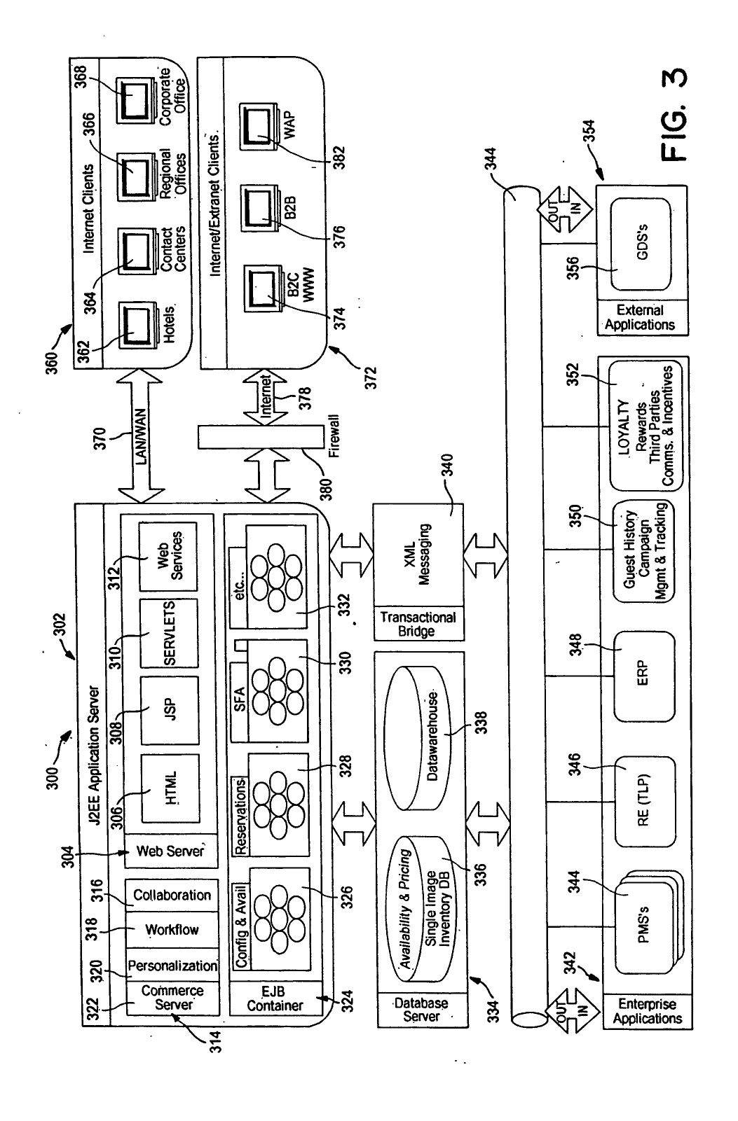

Central interface 250 provides middleware layer interfacing between the centralized inventory system 224 and several components of the hospitality management system, such as business entities 202, distribution channels 230, and revenue management engine 262, using a publish/subscribe messaging system. Other components (see FIG. 3) of the system 200 are also interfaced to the centralized inventory system 224 by means of Central interface 250. Central interface 250 comprises a plurality of interface modules 266 that provide publish/subscribe communications between the various components (e.g., centralized inventory system 224) of the hospitality management system 200. As illustrated, each of the distribution channels 230 are interfaced with the central inventory system using a corresponding module 266. Similarly, the business entities 202 are also each interfaced with the central inventory system using modules 266. Also, revenue management engine 262 and data warehouse component 268 communicate with the centralized inventory's 224 allocation logic module 228 via the publish/subscribe messaging system of module 266. Publish/subscribe communications are asynchronous, multicasting communications and are able to quickly adapt in a dynamically changing environment. The multicasting aspect allows a publisher to send the same event to many subscribers using only one publish operation. The central interface 250 middleware layer may be implemented using Tuxedo™, a product of BEA Systems, Inc. of San Jose, Calif., or other suitable tools.

The dynamic and highly scalable nature of the publish/subscribe communications infrastructure provides a flexible and adaptable technology infrastructure for the hospitality management system 200, facilitating the integration of additional distribution channels 230 and business entities within system 200. While the current publish/subscribe model provides a solution for interfacing and managing the flow of data between different components of system 200, alternative data communication and interfacing architectures may be incorporated within the system 200 without departing from the spirit and scope of the present invention, in contrast, to conventional approaches involving differing procedures (data formats, protocols, etc.) for the various components of existing prior art system, and minimal flexibility and scalability.

Data warehouse 268 comprises a database or repository for storing data on customer requirements and/or preferences, based on data extracted from their prior booking histories. For example, if a customer such as a group 256, or an individual 252, has previously made a booking, the central repository 226 will have stored data on the specific preferences and requirements of that particular customer (i.e., individual or group). Preference information may be, for example, newspaper of choice, room features (e.g., king size bed, smoking, ocean view), fitness facilities, golf courses, etc. The logic control module 228 sends the data from repository 226 to data warehouse 268 via one of the interface modules 266 within central interface 250, where it is stored for further processing by business intelligence module 270.

Business intelligence module 270 processes the data associated with various transactions that may include channel 230 or customer information in order to extract preference/requirements information from the stored information in data warehouse 268. Once the preference/requirements data is extracted by module 270, it is accessed by users within the marketing, sales, or operations business functions of the hospitality organization in order to improve the profitability of marketing efforts and to improve guest satisfaction. Alternatively, the preference/requirements data may be stored in data warehouse 268, where it is accessed by logic control module 228 during the processing of customer requests. The processing of data by the various subsystems of the hospitality management system 200, namely the centralized inventory system 224, central interface 250, revenue management engine 264, data warehouse 268, business intelligence module 270, and the business entities 220, 222, is in real-time. It is in real-time in the sense that information and status information is immediately updated and appropriately stored in different parts of the system once the information or status information changes or becomes available and subject to the ordinary processing and transmission latencies of computer systems and networks. This is particularly the case for the centralized inventory system 224. In effect, there are no associated delays that would likely have a material effect on business decision making.

FIG. 3 illustrates an embodiment of the overall technology architecture 300 of the hospitality management system in accordance with an embodiment of an aspect of the present invention. The technology architecture comprises a multi-tier layered approach running on a server 302, e.g. a J2EE Application server or other suitable server. The first layer comprises a web server 304, which includes Hyper-Text Mark-up Language 306, Java Server Pages™ (JSP) 308, Servlets™ 310, and web services 312 J2EE components. For each of those components, other suitable technology could also be used. Other standard Web Logic tools 314 incorporated in the first architectural tier are: collaboration 316, workflow 318, personalization 320, and commerce server tools 322. For example, the commerce server tools 322 are used for developing on-line store facilities, and the personalization tools are for web security development purposes.

The second tier in the architecture is the container 324 (e.g., Enterprise Java Beans™ (EJB) container), which comprises configuration & availability 326, reservations 328, and sales force automation (SFA) 330 components. These components, implemented according to known methods, form the underlying business rules logic that are applied at the application layer of the architecture. For example, the relevant business rules that are applied to reservation information entered via the application program interface (API) (see screen-shot Figures) are processed by reservations component 328. Similarly, information entered via the API by the hospitality organization's sales team is processed by the SFA 330 component or module. Component 332 illustrates that more components (e.g., EJB or other suitable technology) can be added in order to expand upon the business rules functionality of the system.

The third tier comprises database server 334, which may be an Oracle® database or use other suitable available technology. Database server 334 comprises repository database 336, which is the storage and data management facility for the central inventory system 226. Database server 334 also comprises database 338, which is the data warehouse described in relation to FIG. 2. The third layer in the architecture also includes an XML messaging module 340 for providing communication means between both application server 302, database server 334, and enterprise applications 342. As previously described, interfacing is provided by the middleware architecture of central interface 344. It should be appreciated that the illustrated architecture is not limited to the presently incorporated technologies, but may include, for example, other application interfaces running on a communication network. Also, the communication means may be on a local, metropolitan, or global scale, and utilize wireless (e.g., microwave, satellite, etc.) or waveguide (coaxial cable, fiber optic, etc.) based communication mediums. Moreover, the protocols for information interchange may also vary, based on the organization's size and business needs.

Enterprise applications 342, which communicate with the multi-tiered hospitality managements system via central interface 344, comprise property management systems (PMS) 344, revenue management engine (TLP) 346, enterprise resource planning 348, guest history management 350, and loyalty awards and incentives 352 modules. Each property management system 342 is responsible, among other things, for managing data that is related to reservations, housekeeping, human resources, purchasing, administration, accounting, and facilities of a business entity, such as a hotel. The facilities may include the cafe, restaurant, gift shop, bar, golf course, fitness center, banquet and conference room, or other resources available to the business entity for generating revenue. Once a customer has confirmed a booking or reservation, information related to the booking is sent by the repository database 336 to property management system 344 over XML messaging module 340 and central interface 344. The property management system 324 of the hotel is then updated with the customer's particular room reservation. Similarly, when the customer checks out or does not appear for check-in, such updated status information is sent back to the repository 336 via the interface 344 and XML messaging 340. The repository 336 of the centralized inventory system is thus updated in real time, whereby the room which was blocked as a result of the subsequent booking is released and made available in repository 336.

Revenue management engine 346 receives data from repository 336 over XML module 340 and central interface 344 in order provide yield management calculations. Once booking rates and yield management data is processed by the revenue management engine 346, the data is sent over XML module 340 and interface 344 back to repository 336, where the information is updated in real time. Enterprise resource planning (ERP) module 348 is also updated by information sent from the centralized inventory system's repository 336. The ERP 348 is the hospitality organization's administrative system, which includes for example, among other things, human resources information, financial information, and supply chain information.

Guest history and campaign tracking module 350 holds information about guest preferences, customer lifetime production, promotional productivity, and quantitative measures on marketing efforts concerning the hospitality system's activities. This module is also updated by the centralized inventory system via the messaging module 340 and the central interface 344. The loyalty awards and incentives module 352 keeps track of the organization's various awards programs, commissions, and incentives programs, which encourages individuals and/or organizations to purchase bookings and reservations with the hospitality organization as opposed to another organization. For example, an individual may sign-up or subscribe to receive points for each reservation or booking, which may entitle them to future promotions, special offers, or discounts that are available only to such subscribed customers. Other incentives may include programs that are also available to corporate assistants who book or reserve rooms and/or facilities on behalf of their corporate colleagues or bosses.

External application 354 comprises Global Distribution (GDS) System 356, which as described in relation to FIG. 2, provides a distribution channel for allowing various travel agencies, or third parties, to access and make real time enquiries, bookings, and reservations on behalf of customers by sending their queries to the centralized inventory system for processing. As illustrated, these GDS's 356 also communicate with repository 336 over XML messaging module 340 and central interface 344.

The organization's internet clients 360 comprise hotels 362, contact centers 364, regional offices 366, and corporate offices 368, which communicate with the application server 302 of the hospitality management system 200 over the organization's local area network and/or wide area network resources 370. For example, the organization's sales force may gain access to the application server 302 and the relevant web pages for making bookings for group customers through one of the internet clients 360. Similarly, external internet clients 372 comprise Business-to-Business 376 (B2B) and Business-to-Consumer (B2C) 374 clients that can access the hospitality organization's application server 302 over the internet 378, and via the hospitality organization's firewall 380. Wireless Application Protocol (WAP) tools 382 may also be used to provide wireless access to the internet and thus the application server 302.

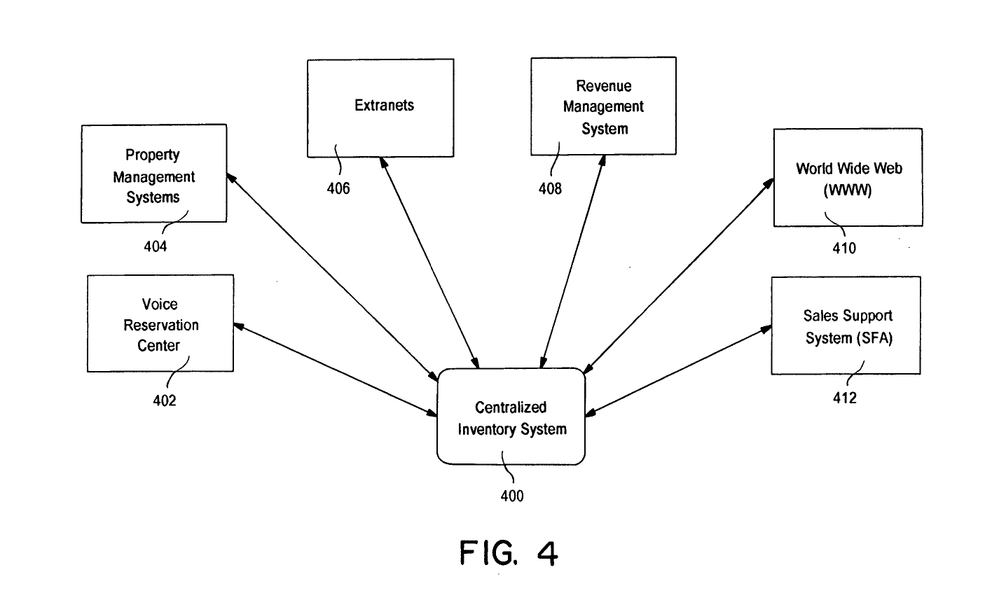

In an embodiment of an aspect of the present invention, FIG. 4 shows a conceptual illustration of the interaction of the centralized inventory system 400 with other entities, both within the hospitality management system and external to the hospitality management system. The centralized inventory system bi-directionally communicates with a voice reservation center 402, property management systems 404, extranets 406, the revenue management engine 408, internet based clients 410, and a sales support or sales force automation (SFA) system 412. Voice reservation center 402 provides the customer with an opportunity to dial a central toll-free number in order to make reservation enquiries and bookings. Rates are proposed by the centralized inventory system 400 and communicated back to the voice reservation center 402. All bookings for all business entities within the organization are established through the centralized inventory system 400.

The property management systems (PMS) 404 associated with respective business entities communicate status information associated with the rooms and facilities, and do not set room rates or pricing proposals based on customer queries. The centralized management system 400 informs the PMS 404 of a particular business entity of any bookings that have been made by customers (i.e., confirmed bookings). The PMS 404, in turn, informs the centralized inventory system 400 of status updates that relate to the booked facilities or rooms being vacated or made available for new business (e.g., check-out). Extranets 406 are developed for clients that are given special access to a particular application interface within the organization's application server 302 (FIG. 3). For example, the hospitality organization will create a specific internet web page that provides exclusive bookings for employees of a company. The company is given password access to the extranet 406, where booking rates for room and/or facilities are calculated based on previously negotiated terms and conditions of a contract between the hospitality management system and company.

Revenue management engine 408 receives data from the centralized inventory system 400 and calculates rates and applies inventory restrictions in order to maximize revenues. Once a rate proposal has been calculated by the revenue management engine 408, the data associated with the generated rate proposal is sent to the centralized inventory system 400, where it distributes the rate proposal to the required channel, such as, for example, voice reservation center 402, or an extranet 406 client. Centralized inventory system 400 also centrally processes reservations and bookings for internet based clients 410. The internet based clients 410 may include customers that access the hospitality organization's reservations and bookings webpage (or other user interface) via the organization's homepage. The customer's proposal request (i.e., a booking or reservation quote) for a particular booking is sent to the centralized inventory system 400, where it is processed. The calculated rate is then sent from the centralized inventory system 400 back to the customer. Other internet-based clients may be on-line merchants, which use B2B internet access to provide customers with rate proposals from the centralized inventory system 400.

The hospitality organization's sales force system 412 provides distributed sales operations via the Internet from hotels 362 (FIG. 3) and corporate offices 368 (FIG. 3), providing real-time room availability. As with the other systems, the sales force automation system 412 sends rate or pricing queries to the centralized inventory system 400, which in turn generates rates or pricing proposals that are sent back to the relevant agent via the sales force automation system 412. The sales force automation system 412 comprises a sales force automation component 330 (FIG. 3), which provides corporate negotiations and tracking for individual customers, groups (e.g., tourist groups) and events (e.g., conferences) by account. For example, the sales force automation (SFA) component 330 (FIG. 3) may provide an agent with an email reminder to follow-up on a prior negotiated contract with a particular group. If a block of rooms were only tentatively booked for this group and not finalized, the rooms may be released by the centralized inventory system. The reminder allows the agent to follow up with the group in order to ensure that the rooms they had tentatively requested are not lost. The reminder also enables rooms and resources to be released back into the inventory if, after the follow-up, the group no longer wishes to proceed with finalizing the booking. Using the SFA component 330 (FIG. 3) of the system, agents can handle bookings for multiple business entities without having to negotiate with a manager at each business entity. The centralized inventory system 400 provides a proposed rate, which is uniform across all other channels of distribution (i.e., sales), which provides the customer with a higher level of confidence as to the “fairness” of the proposed pricing.

The sales force automation module 330 (FIG. 3) is comprised of underlying business rules for processing and managing the reservation or booking steps for groups. Examples of such business rules may include follow-up dates, alternative dates for a particular booking based on a given rate, cut-off dates for payment of deposits, multiple business entity enquiry capabilities, rates based on length of stay, etc. The sales force automation module 330 (FIG. 3) uses the revenue management engine to generate real time recommendations for determining rates that can be quoted by the hospitality organizations sales agents in generating a proposal. This rate includes “loose-it” recommendations, which is a minimum rate that can be offered to the groups based on their request for rooms or facilities. For example, if 100 rooms were requested at $100 each per night, the revenue management engine may forecast that these rooms should not be sold at these rates, as revenues can be maximized by an anticipated seasonal increase in transient tourists that will pay a much higher rate for the rooms.

If a group's request for a particular rate is rejected following the revenue management engine's calculations, the sales agent may contact a revenue manager or the revenue management team, which is a person or group of people responsible for overseeing revenue management within the hospitality organization. The revenue manager has the authority to override the revenue management engines recommendations that are generated as part of the centralized inventory system's processing of potential bookings.

The sales force automation module 330 (FIG. 3) manages prospective bookings or pricing prospects for groups (i.e., “leads”), and provides the sales agent with various rules and policies governing the groups' stay at the hotel or particular business entity. For example, some large corporations wishing to host conferences or trade shows at a particular business entity of a hospitality organization, may request that non-alcoholic drinks be served. Alternatively, they may request (in the contract) certain size rooms for their company executives. This and other similar information is handled by the sales force automation module 330 (FIG. 3) for assisting the hospitality organization's sales agents in effectively managing group bookings or reservations.

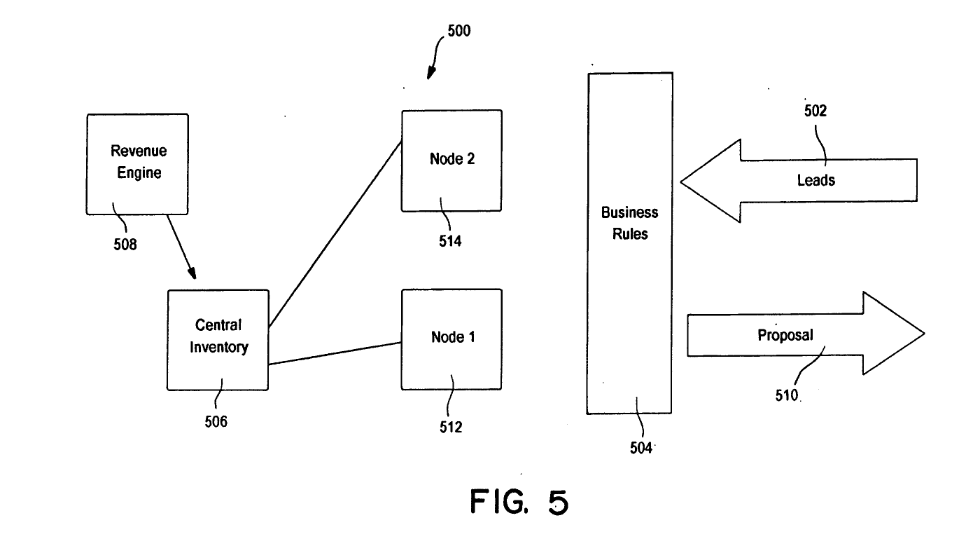

In an embodiment of an aspect of the present invention, FIG. 5 illustrates the flow of data between sales agents or executives of the hospitality organization and the hospitality management system 500, for both individual and group bookings and reservations. The sales agents send prospective queries 502 (i.e., leads) regarding a group booking to hospitality system 500 over the web according to business rules 504. Business rules 504 for “leads” may include, among other things, specifying the length of stay, alternative dates of interest, etc. Data associated with a “lead” is sent to the centralized inventory system 506 for processing, where the revenue management engine 508 receives this data from the inventory system 506 and performs a group analysis. Once the analysis is complete, the centralized inventory system 506 sends a proposal 510 associated with the lead to the sales agent according to business rules 504. For example, according to the business rules 504, a cut-off date will be set for responding to proposal 510. If appropriate payments are not received by the system before this date, the requested resources specified in the “lead” are released, and become available to all other groups or channels. Sales entities, in contrast to sales agents, can be sales staff, personnel of a business entity such as a property of a hotel, travel agent, event planner, without limitation.

The revenue management engine 508 generates a variable metric know as “booking points” in response to processing received leads from the sales force. Depending on the booking points a group receives, the revenue managers can reject or accept a group's booking. For example, if two groups are competing for resources within a particular business entity, the revenue managers may use the booking points as a means of determining which group to reject. It will be appreciated, however, that the revenue managers, who in an embodiment of an aspect of the present invention possess the authority to override the revenue management engine 508, have the discretion of rejecting a group with higher booking points, if it is determined that the group with lower booking points is a potentially important future customer.

The business rules. 504, which are applied to the hospitality management system 500, are run over several nodes, such as nodes 512 and 514, which may form a J2EE cluster. As illustrated, both nodes 512, 514 support the operation of the centralized inventory system 506. The nodes 512, 514 optimize database access and provide added redundancy for managing the real-time data transactions between sales channels and the centralized inventory system 506.

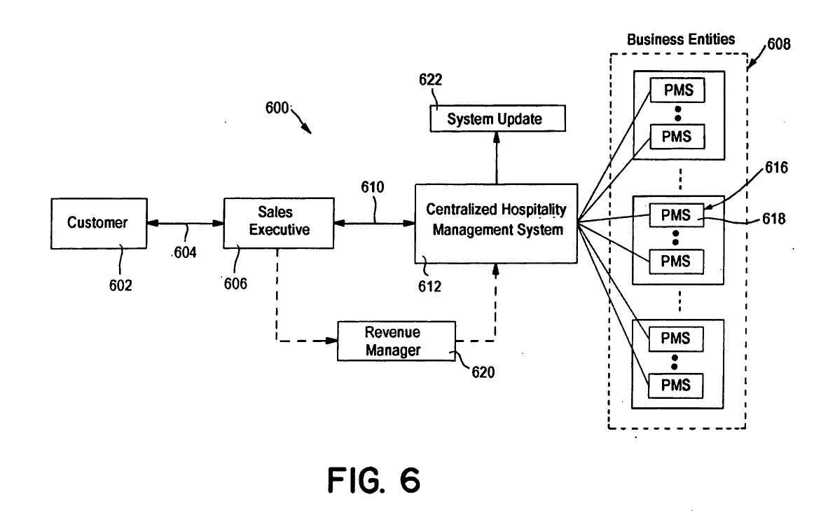

FIG. 6 illustrates a simplified diagram showing the basic communication of data between the various components of the hospitality management system 600. Customer 602 requests a quote (e.g., a booking, pricing proposal or the like) by contacting a sales executive or agent 606 (who may refer to it as a “lead”) over communication channel 604. It will be appreciated that the customer may use any other available channel, as well as the sales agent, for requesting a quote. The communication link 604 could be, but is not limited to, email, Internet, facsimile, or other means of communication. The sales agent 606 generates a “lead” based on the customers requests and preferences for a particular group booking at one of the business entities 608. This lead is sent via communication link 610 to the centralized hospitality management system 612. The centralized hospitality management system 612 comprises the multi-tiered architecture of the application server, EJB containers, and database server described in FIG. 3. In the current embodiment, communication link 610 is the Internet, however, the communication link may include any compatible communications solution between the sales agent 606 and centralized hospitality management system 612. If the lead is accepted by centralized hospitality management system 612 following the real-time processing and analysis of the revenue management engine (not shown), a proposal is sent back over link 610 to sales agent 606. If the customer 602 also accepts the proposal, the required facilities are reserved at one of the business entities 608 of choice, such as, business entity 616. Consequently, the centralized hospitality management system 612 sends the booking or reservation information to the business entity's property management system 618 (PMS).

If the lead is not accepted by the centralized hospitality management system 612, the sales agent 606 may contact revenue manager 620 and propose that the lead be accepted based on the circumstances associated with the group requesting the booking (e.g., potentially large future customer, or future business partner, etc.). If the proposal of agent 606 is accepted, the revenue manager 620 (which may be a person) may manually override the centralized hospitality management system's 612 rejection of the group's lead, and enter the booking into the system. All relevant information regarding bookings and resource allocation are distributed by the centralized hospitality management system 612 to other system components for updating 622 (e.g., updating of ERP, updating earned Loyalty Rewards based on bookings).

In FIGS. 2 through 7, although certain entities may be identified by differing reference numerals in the various figures for purposes of illustration, they may nevertheless correspond to the same entity in embodiments of the invention.

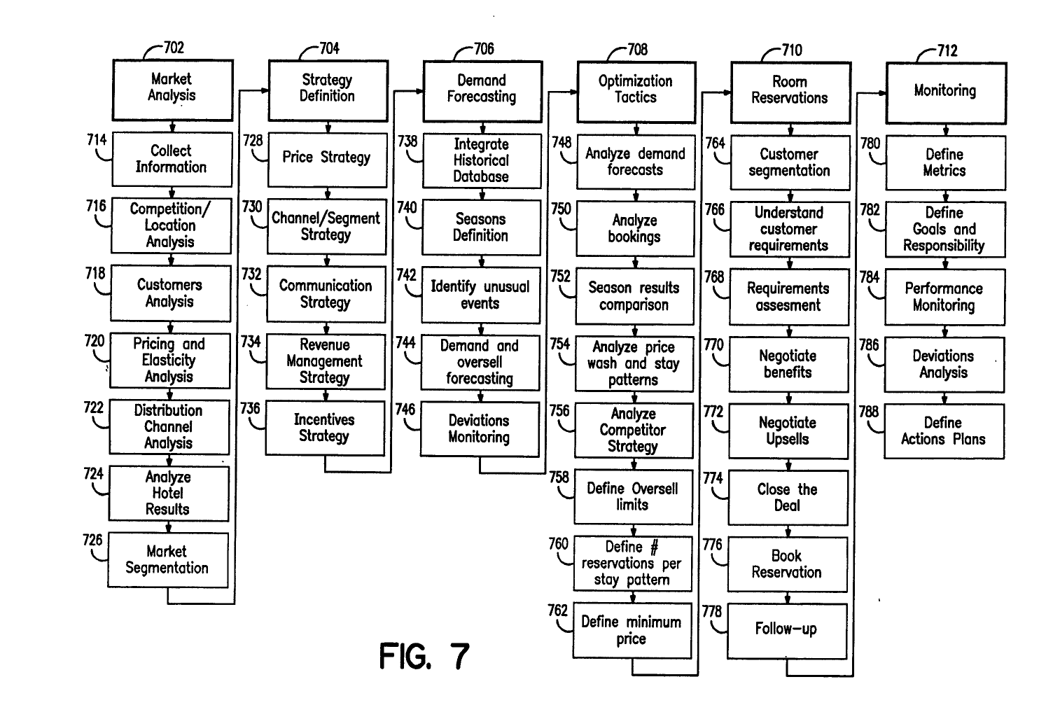

FIG. 7 illustrates the information processing used by the hospitality management system in processing customer queries and generating pricing proposals for various facilities within the business entities, according to an embodiment of an aspect of the present invention. The hospitality management system processes various parameters that are associated with the hospitality industry. According to the present embodiment of the invention, these processes are categorized into market analysis 702, strategy definitions 704, demand forecasting 706, optimization tactics 708, room reservations 710, and monitoring 712. Each category includes a number of phases or steps that are attended to or executed according to the arrows. The ordering of these steps, which may vary without departing from the invention, are discussed below.

Marketing analysis 702 comprises the assessment of information such as, information collection 714, competition/local analysis 716, customer analysis 718, pricing and elasticity analysis 720, distribution channel analysis 722, analysis of hotel results 724, and market segmentation 726.

Strategy definition 704 category comprises attending to price strategy 728, channel/segment strategy 730, communication strategy 732, revenue management strategy 734, and incentives strategy 736.

Demand forecasting category 706 involves integrating historical database 738, identifying seasons definitions 740, identifying unusual events 742, demand and oversell forecasting 744, and deviations monitoring 746.

Optimization tactics 708 includes analyzing demand forecasts 748, analyzing bookings 750, seasons results comparisons 752, analyzing price wash and stay patterns 754, analyzing competitors strategy 756, defining oversell limits 758, defining number of reservations per stay pattern 760, and defining a minimum price 762.

Room reservation category 710 comprises customer segmentation 764, understanding customer requirements 766, conducting requirements assessment 768, negotiating benefits 770, negotiating up-sells 772, closing deals 774, booking reservations 776, and carrying out follow-ups 778.

The monitory category 712 includes defining metrics 780, defining goals and responsibilities 782, conducting performance monitoring 784 and deviations analysis 786, and defining action plans 788.

Information collection 714, competition/local analysis 716, customer analysis 718, pricing and elasticity analysis 720, price strategy 728, and communication strategy 732 are processes that are executed as part of the hospitality organization's sales methodology. Integrating historical database 738, identifying seasons definitions 740, identifying unusual events 742, demand and oversell forecasting 744, deviations monitoring 746, analyzing demand forecasts 748, analyzing bookings 750, seasons results comparisons 752, analyzing price wash and stay patterns 754, defining oversell limits 758, defining the number of reservations per stay pattern 760, and defining price minimum 762 are processes that may be executed by the hospitality organization's revenue management process (i.e., revenue management engine 262 shown in FIG. 2). Analyzing bookings 750, conducting seasons results comparisons 752, analyzing price wash and stay patterns 754, performing customer segmentation 764, understanding customer requirements 766, engaging in requirements assessment 768, negotiating benefits 770, negotiating up-sells, booking reservations 776, carrying out follow ups 778, performance monitoring 784, and conducting deviations analysis 786 are processes that may be executed by the centralized inventory system 224 (FIG. 2).

Distribution channel analysis 722, analysis of hotel results 724, market segmentation 726, channel/segment strategy 730, revenue management strategy 734, incentives strategy 736, analyzing competitors strategy 756, closing the deals 774, defining metrics 780, defining goals and responsibilities 782, and defining action plans 788 are processes that may be performed manually.

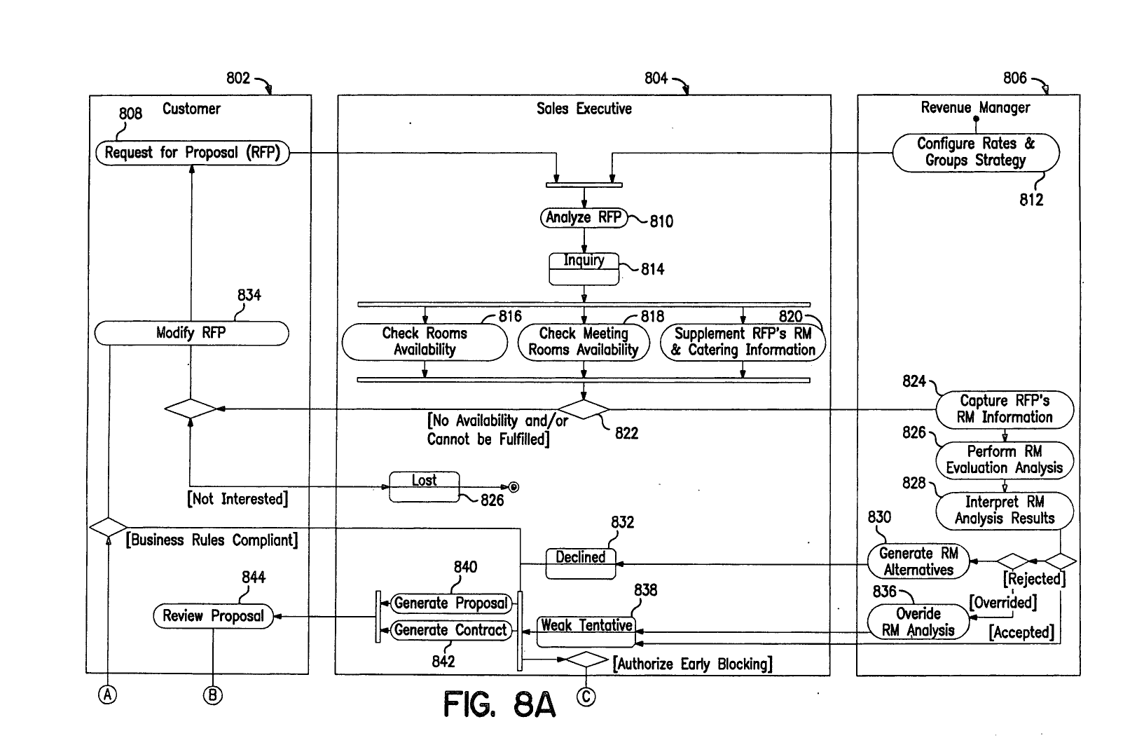

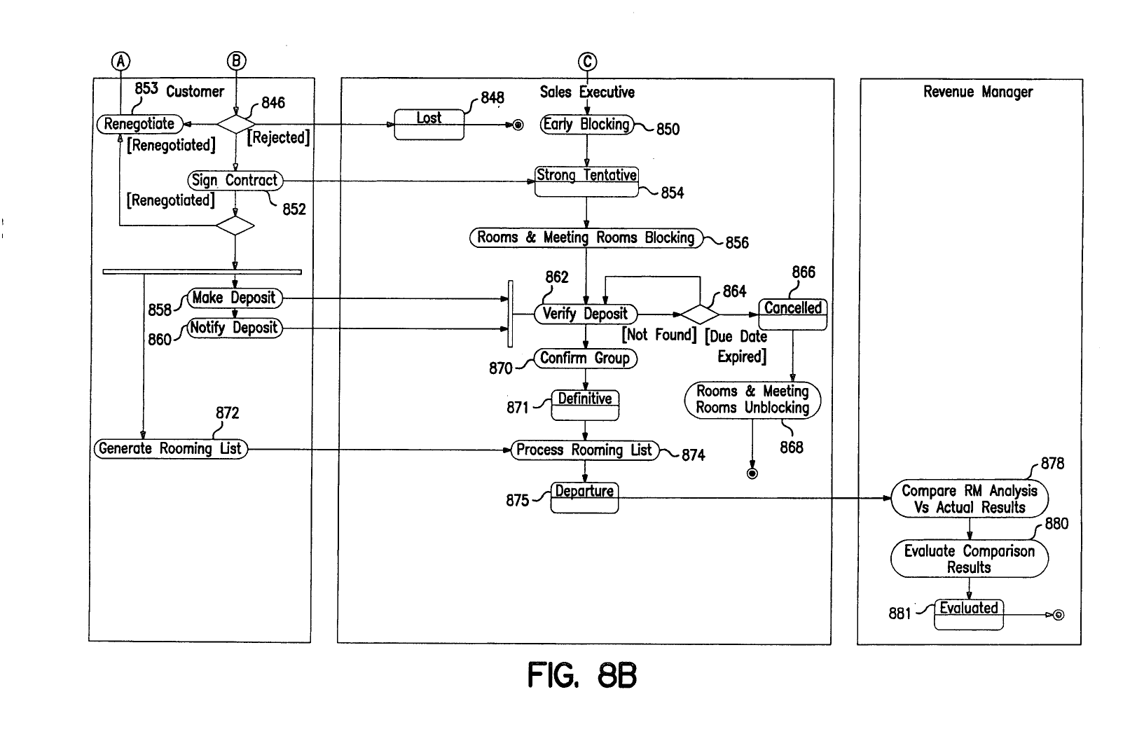

FIG. 8A illustrates a flow diagram of the business processes associated with the hospitality management system in an embodiment of an aspect of the present invention. The business process steps are between a customer 802, a sales executive or agent 804, and a revenue manager 806. The revenue manager specified in the context of the descriptions of FIGS. 8A and 8B can correspond to both a revenue manager, which is a person, or a revenue management engine, which performs revenue management by processing data associated with the customer's requests.

At step 808, a customer makes a request for proposal or quote, which may include information associated with the required facilities, e.g., number of rooms, the length of stay, and catering, conference rooms etc. At step 810, the request for proposal or quote generated by the customer is analyzed by the sales agent. Configuration rates and group strategy information 812 is also accessed by the sales agent for analyzing the request for proposal at step 810. Following the analysis of the proposal, at step 814, an inquiry for the request for proposal is generated. At steps 816, 818, and 820, the generated inquiry is processed in order to determine room availability, meeting room availability, and/or the availability of the facilities. If at step 822 it is determined that the requirements of the request for proposal inquiry are met, at step 824 the revenue management information associated with the received request for proposal is captured for analysis. The revenue manager may access price setting data associated with the proposal from the centralized inventory system. At step 826, revenue management evaluation analysis is carried out (i.e., revenue management engine), and at step 828 the results of this analysis is interpreted. Based on the results of the interpreted revenue management evaluation analysis, carried out at step 828, the proposal is either rejected or accepted.

If the request for proposal is rejected, at step 830 the revenue manager generates alternative proposals to the customer's request. At step 832, it is determined that the customer's request is declined, and based on the revenue manager's generated alternative proposals, at step 834 a modified request for proposal is sent back to the customer for consideration.

If the request for proposal is accepted following steps 826 and 828, based on a weak tentative prospect 838 of finalizing the booking or reservation, at steps 840 and 842, both a proposal and contract are generated based on the customers request.

Alternatively, when the request for proposal is rejected, at step 836, the sales agent may contact the revenue manager and request that the proposal be accepted. If the revenue manager decides that there is grounds for accepting the proposal (e.g., potentially important customer, such as a corporation), the evaluation analysis and its interpretation (generated by the revenue management engine at steps 826 and 828) are overridden. Based on a weak tentative prospect 838 of the booking being finalized, as indicated at steps 840 and 842, both a proposal and contract are generated based on the customers request.

If at step 822 it is determined that the requirements are not met (e.g., no room availability), at step 834 the request for proposal is modified and sent back to the customer at step 808. If at step 808 the customer accepts the modified proposal, the proposal will be resent to the sales agent for analysis at step 810. If the request cannot be fulfilled and the customer is not interested in pursuing a new request for proposal, at step 826, the request for proposal is terminated.

Once a proposal and contract has been generated at steps 840 and 842, respectively, at step 844 the proposal is sent to the customer for review. The subsequent steps in the method illustrated in FIG. 8A are shown in FIG. 8B. As illustrated in FIG. 8B, once the customer receives the proposal, at step 846, they may reject, accept, or renegotiate the terms of the proposal (e.g., price of rooms). Also, concurrently with sending the proposal to the customer, as indicated at step 844, the sales agent is authorized to make an early conditional blocking of the requested rooms, as indicated at step 850. If the prepared proposal is accepted, at step 852, the customer has the option to sign a contract. This indicates a strong tentative prospect of finalizing the booking, as indicated at step 854. If the customer signs the contract, at step 856, the sales agent blocks the rooms and resources specified in the generated proposal. However, at step 846, the customer may reject the generated proposal that was sent for the customer's review at step 844 (FIG. 8A). In this case, the booking process is terminated, as indicated at 848. Alternatively, at step 853, the customer may decide to renegotiate the terms of the proposal. At step 834 (FIG. 8A), the customer's request for proposal is modified and resubmitted by the sales agent for reevaluation (i.e., steps 808, 810, 816, 818, 820, 824, 826, 828). Also, at step 852, the customer might not sign the contract, but might decide to renegotiate the terms of the generated proposal, as indicated at step 854.

Once the contract has been signed, the rooms and resources (i.e., facilities) specified in the generated proposal or quote are blocked, as indicated at step 856. At steps 858 and 860, once a deposit has been paid and a deposit notification is sent to the sale agent, at step 862, the deposit must be verified. At step 864 the due date for receiving payment of the deposit is checked. If this date has expired, as indicated at step 866, the rooms and resources that were blocked are released, as indicated at step 868. If the deposit is verified, the group booking is confirmed, as indicated at step 870. This indicates that the booking status is definitive, as indicated at step 871. Therefore, the rooms or resources (i.e., facilities) are fully allocated to the customer. At step 872, a rooming list (i.e., a list of persons occupying rooms) is generated as a result of the confirmation in step 870. Once the generated rooming list is generated, it is processed at step 874. At step 875, once the departure of the customer or group is established, at step 878, the results of the actual confirmation (i.e., actual revenue generated) are compared with the revenue manager's analysis. At step 880, the results of this comparison is evaluated for further revenue management planning and consideration. At step 881, the evaluation status is determined to be complete.

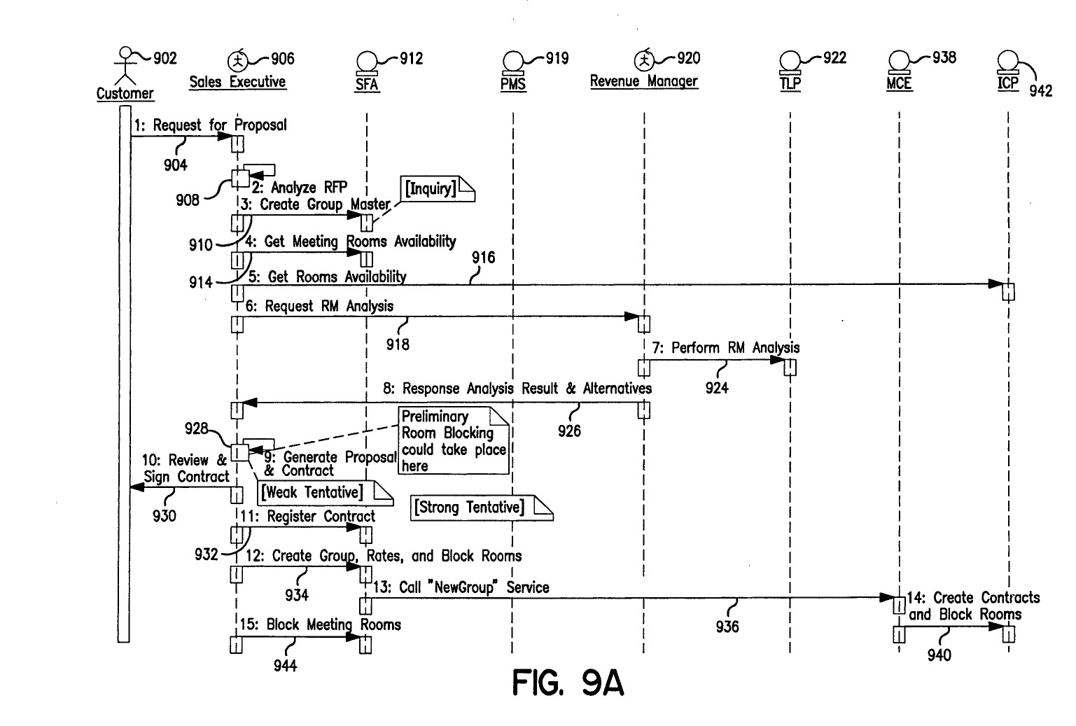

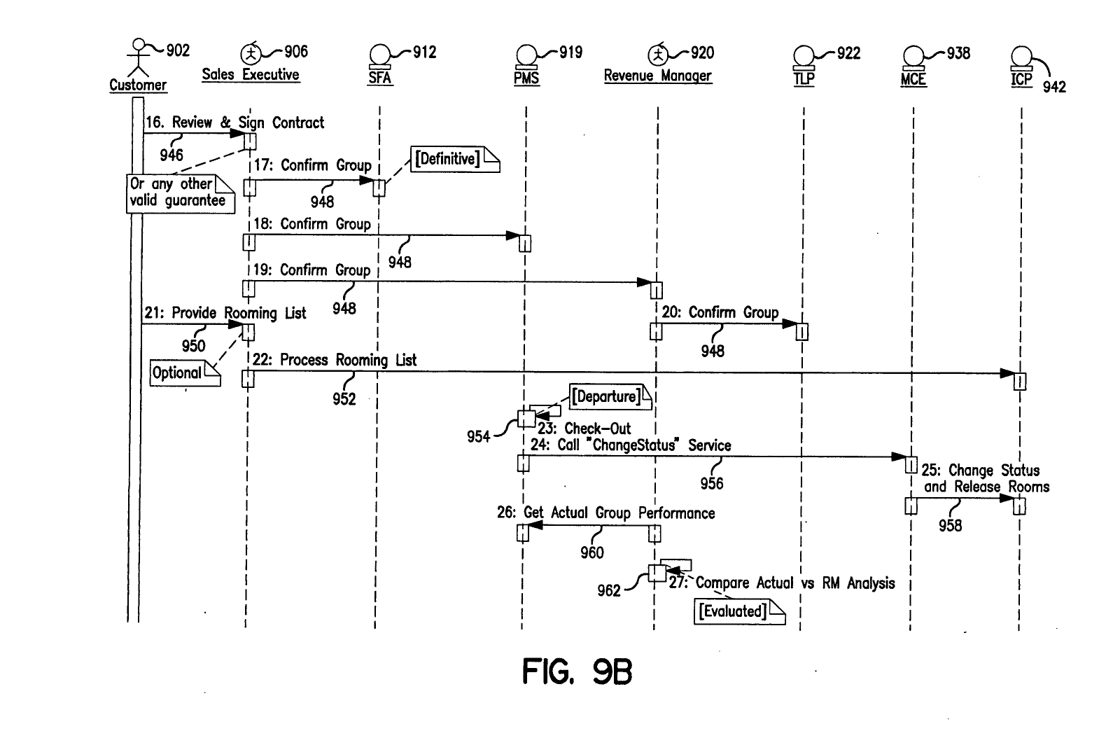

FIG. 9A illustrates an alternative flow diagram of the methods associated with the hospitality management system in an embodiment of an aspect of the present invention, wherein the process steps associated with FIGS. 8A and 8B are indicated in relation to the customer, the sales agent, and the different components of the hospitality management system.

Customer 902 makes a request for a pricing proposal 904 from sales agent 906. The sales agent 906 analyzes the request for proposal 908. The sales agent 906 then creates a group master 910 (inquiry) based on the request for proposal, which is sent to the sales force automation module 912 (FIG. 3). Meeting rooms availability 914 is determined at sales force automation module 912 by the sales agent 906. Also, the sales agent 906 checks room availability 916 by accessing centralized inventory system 942. Once availability has been determined at the centralized inventory system 942, the sales agent 906 requests revenue management analysis 918 from the revenue manager 920. If no negotiation with the revenue manager 920 is required, revenue management analysis and processing 924 is performed on the inquiry associated with the request for pricing proposal by means of revenue management engine 922.

The results of the analysis performed by revenue management engine 922 and proposed alternatives to the inquiry 926 are sent by revenue manager 920 to sales agent 906, where a proposal contract is generated. Preliminary room (or other facility) blocking may also occur at this stage. The proposal contract is then sent to customer 902 for customer review and signing 930. The proposal contract contains the price quote associated with the customer's 902 request for a pricing proposal. Once the contract is signed, sales agent 906 registers the contract 932 with the sales force automation module 912. Following registering the contract 932, information on the group, rates, and rooms to be blocked 934, are created and sent by the sales agent 906 to the sales force automation module 912. The sales force automation module 912 calls (i.e., call “NewGroup” service) the publish/subscribe messaging system 936 in the central interface 938 in order for group data to be sent to the revenue management engine 922, centralized inventory system 942, and the property management system 919, so that contract information including blocked rooms 940 is updated. Information associated with blocking the meeting rooms 944 is then sent from sales agent 906 to sales support system 912.

The subsequent steps in the business process illustrated in FIG. 9A is shown in FIG. 9B. As illustrated in FIG. 9B, once customer 902 reviews and signs the contract 946, it is received by the sales agent 906. A valid guarantee, such as a deposit, may be required. However, this requirement may be overridden for important clients. The sales agent 906 then confirms the group booking 948 (i.e., definitive) with sales force automation module 912, PMS 919, and the revenue manager 920. The revenue manager 920 also confirms the group booking 948 with revenue management engine 922. An optional rooming list 950 is sent by customer 902 to sales agent 906, whereby the sales agent in turn sends the rooming list for processing 952 at the PMS 919. Once it has been entered into the PMS 919 that the group has checked out 954 (i.e., departure), the PMS 919 calls a “ChangeStatus” service 956 at central interface 938. Central interface 938 then sends a change of status and information on the released rooms to centralized inventory system 942.

The revenue manager 920 gets the actual group performance 960 from the PMS 919, and compares (i.e., evaluates) the group performance versus the revenue management analysis 962, which was performed by the revenue management engine 922. This provides a measure of accuracy associated with the revenue forecasting carried out by the revenue management engine.

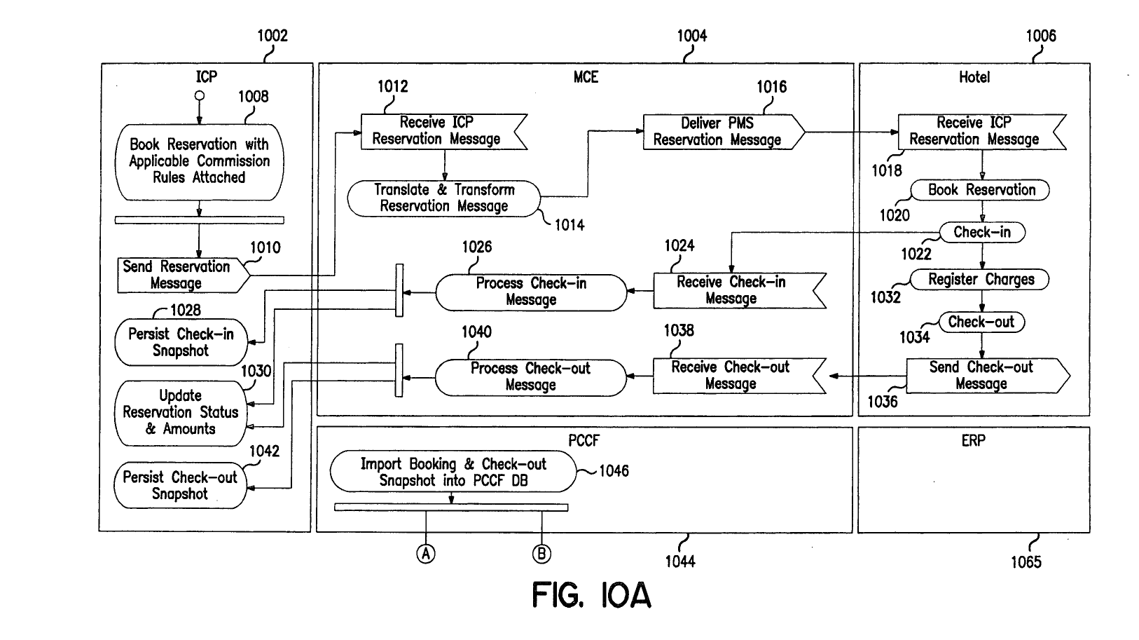

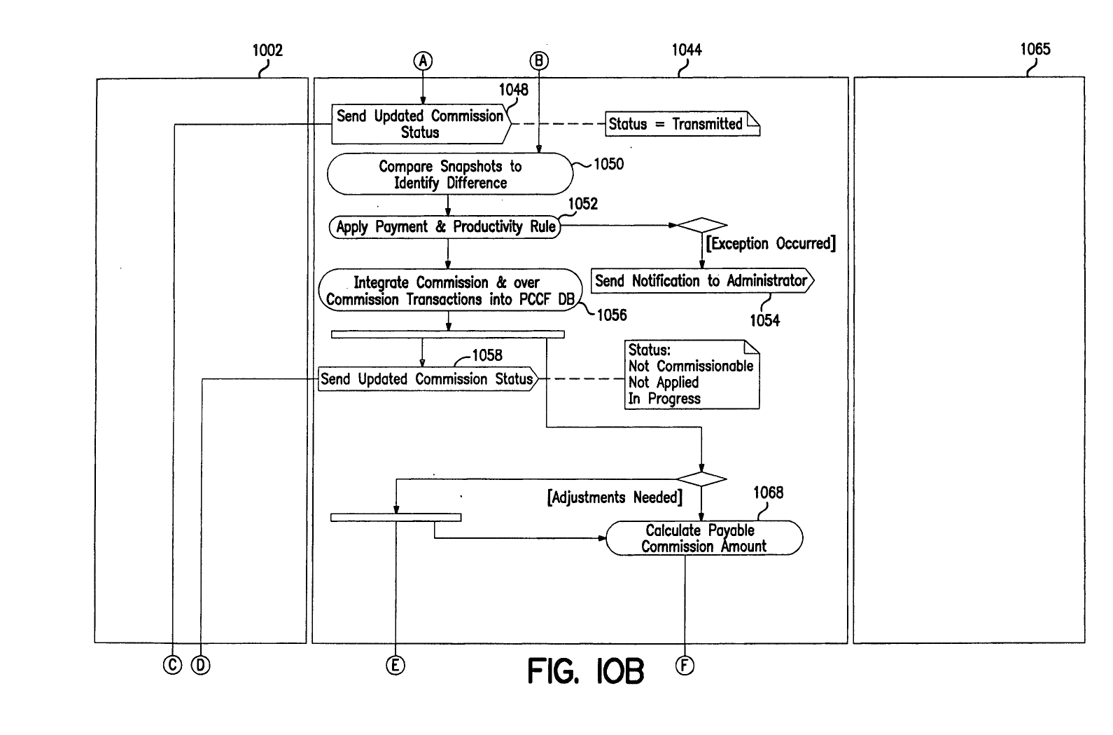

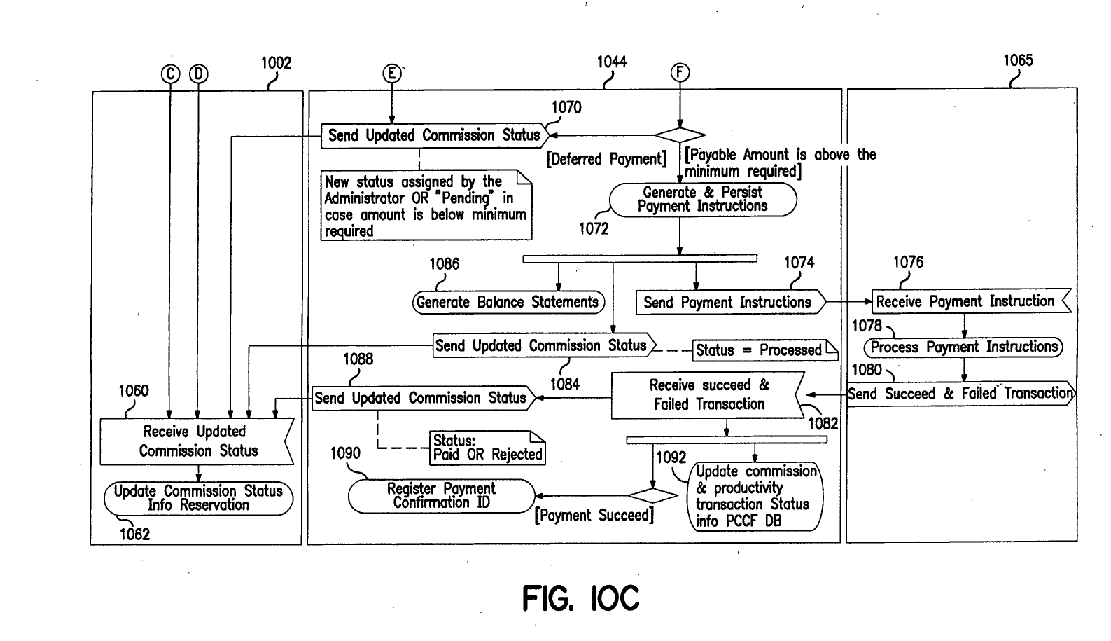

FIG. 10A illustrates a flow diagram for the methods associated with the reservations and the centralized commission payment system of the hospitality management system in an embodiment of an aspect of the present invention. As illustrated, the process steps show interaction between the centralized inventory system 1002, the central interface 1004, and a business entity such as a hotel 1006. At step 1008, the centralized inventory system 1002 enters a booking or reservation with the applicable commission rules attached. The commission rules depend on the distribution channel that was used in establishing the booking. For example, the commission amount paid to a GDS, consortia, or online merchant will vary between them. At step 1010, the centralized inventory system 1002 sends the reservation message to the central inventory 1004. At step 1012, the central interface 1004 receives the centralized inventory system's reservation message, and translates this message into a format allows the property management system (PMS) of the hotel to receive and process the message, as indicated in step 1014. At step 1016, the central interface 1004 delivers the reservation message to the hotel's PMS. Based on the received reservation message, at step 1020 the booking or reservation for the room or rooms is made. Following this booking, at step 1022, check-in information is entered into the hotel PMS. Once check-in is complete, at step 1024, the central interface 1004 receives a check-in message from hotel 1006. At step 1026, the check-in message is processed and sent to the centralized inventory system 1002, where at step 1028, the check-in snap shot (i.e., a copy of the reservation data at check-in) is entered. Based on the received check-in message (step 1026), at step 1030, reservation status and amounts are updated in the centralized inventory system 1002. At the hotel 1006, at step 1032 all charges associated with a customer's stay are registered in the PMS of the hotel. At step 1034, a check-out process is complete, and a message associated with the check-out process is sent to the central interface 1004, as indicated at step 1036. At steps 1038 and 1040, the check-out message is received by the central interface and processed. Based on the received check-out message (step 1040) at step 1030, reservation status and amounts are updated in the centralized inventory system 1002. In this case, however, check-out status is updated in the centralized inventory system 1002. At step 1042, a check-out snap shot is entered into the system 1002.

At the centralized commission payment system 1044, at step 1046, booking and check-out snap shots are imported into the centralized commission payment system's database.

The subsequent steps in the methods illustrated in FIG. 10A are shown in FIG. 10B. As illustrated in FIG. 10B, At step 1048, updated commission status (i.e., transmitted) is sent to the centralized inventory system 1002 for updating the commission status, as indicated at steps 1060 and 1062 (FIG. 10C). At step 1050, the booking and check-out snap shots are compared in order to identify any differences. At step 1052, based on the identified differences, payment and productivity rules are applied. If any exceptions (e.g., mistakes, fraud, inconsistencies in payment amounts, etc.) occur during this process, at step 1054, a notification is sent to the administrator. Following the application of payment and productivity rules (step 1052), at step 1056, commission and over commission transactions are integrated into the centralized commission payment system's database. At step 1058, this updated commission information is sent to the centralized inventory system 1002 for updating the commission status, as indicated at steps 1060 and 1062 (FIG. 10C). The updated status at step 1058 may be “not commissionable,” “not applied,” or “in progress.” At step 1068, the payable commission amount is calculated.

The subsequent steps in the methods illustrated in FIG. 10B are shown in FIG. 10C. As illustrated in FIG. 10C, once adjustments are updated in the database, as indicated at step 1066 (FIG. 10B), at step 1070, updated commission information is sent to the centralized inventory system 1002 for updating the commission status, as indicated at steps 1060 and 1062. Once the payable commission is calculated at step 1068 (Figure B), payments are either deferred, whereby the deferred status is updated at step 1070, and sent to the centralized inventory system 1002 for updating the commission status, as indicated at steps 1060 and 1062. Alternatively, if payments are not deferred, at step 1072, payment instructions are generated. Based on the generated payment instructions indicated at step 1072, payment instructions are sent to the enterprise resource planning (ERP) system 1065, as indicated at step 1074. At steps 1076 and 1078, the payment instructions are received and processed by ERP system 1065. At step 1080, succeeded and failed transaction information associated with the commission payment is sent to the centralized commission payment system 1044, where it is received at step 1082. Following step 1072, based on the generated payment instructions, at step 1084, updated commission status is sent to the centralized inventory system 1002, as indicated at steps 1060 and 1062. Also following step 1072, at step 1086, balance statements are generated.

At step 1088, the succeeded and failed transaction information received from step 1082 is sent to the centralized commission payment system 1044, where the commission status (i.e., paid or rejected) is received and updated, as indicated at steps 1060 and 1062. Following step 1082, if the payment transaction succeeded, at step 1090 a payment confirmation identification (ID) is registered at the centralized commission payment system 1044. Also, at step 1092, commission and productivity transaction status information is updated in the centralized commission payment system's database.

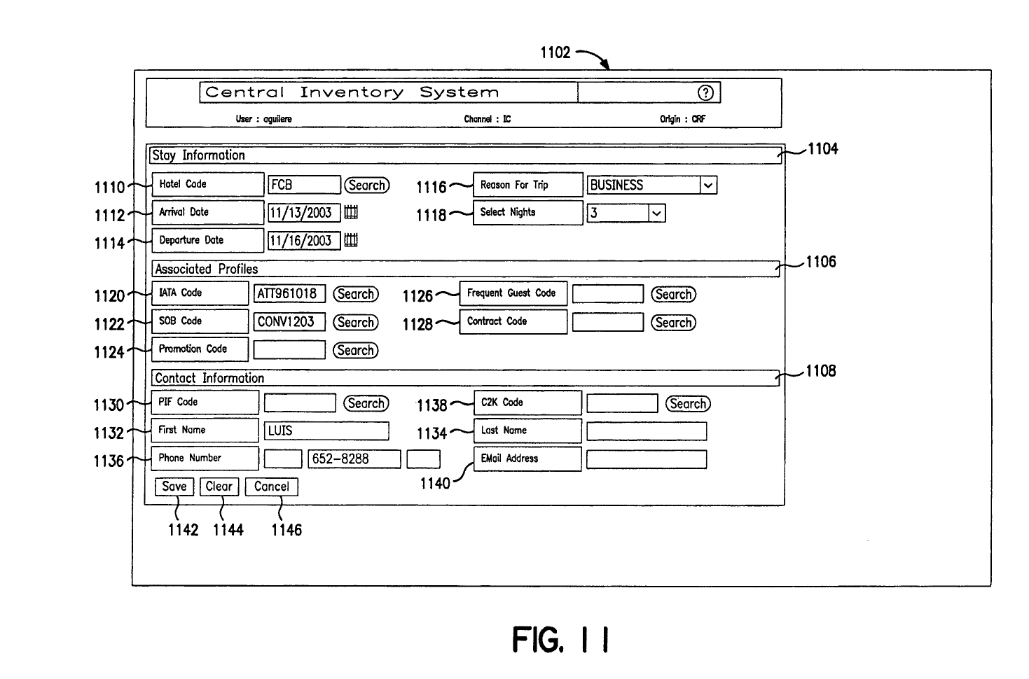

FIG. 11 shows an embodiment of a user interface (here a web-page screen-shot) 1102 for entering stay information associated with reservations made through a call center (FIG. 2, call center 236) in accordance with an aspect of the present invention. As illustrated, the data entry screen comprises a stay information section 1104, associated profiles section 1106, and a contact information section 1108. The stay information section 1104 includes data entry fields for entering a hotel code 1110, arrival data 1112, departure date 1114, reason for trip 1116, and the number of nights for stay 1118.

The associated profiles section 1106 includes data entry fields indicating IATA code 1120, Source of Business (SOB) code 1122, promotion code 1124, frequent guest code 1126, and contract code 1128. Through the IATA code 1120, the hospitality management system manages and processes various commission payments to travel agencies. Based on the SOB code 1122, promotion code 1124, and frequent guest code 1126, the customer is quoted a special rate. The contract code 1128 field may not be filled if there is no contract. If a contract code is entered, the booking rate that is quoted or proposed to the customer will depend on prior negotiations that may have occurred, for example, between the sales agent, revenue manager (person), and customer.

The contact information section 1108 includes data entry fields for entering PIF code 1130, first name 1132, last name 1134, phone number 1136, C2K code 1138, and email address 1140. The PIF code is used for allocating loyalty points to various sales agents that book directly through the hospitality organization's website or central reservation system. Similarly, C2K code 1138 is another loyalty points system for giving company personal assistants (PA) an incentive to make bookings for their colleagues and managers using the hospitality organization's direct booking channels, such as its website or central reservation system. Information entered in screen 1102 can be saved 1142 or cleared 1144. Alternatively, the user may exit screen 1102 by activating cancel 1146.

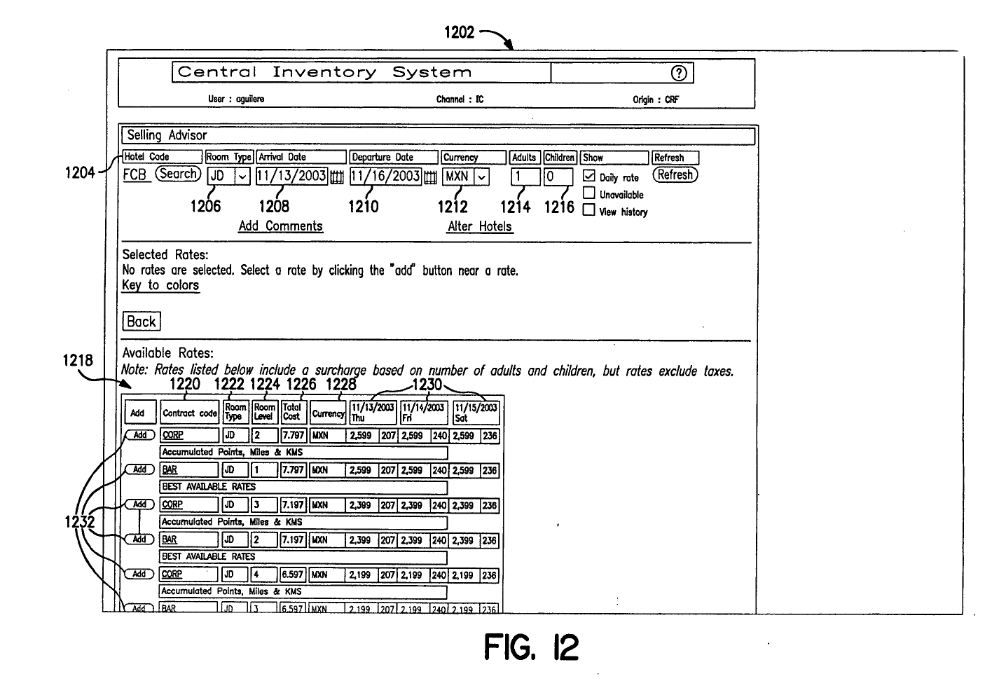

FIG. 12 shows an illustrative example of a web-page screen-shot 1202 for indicating availability information associated with data entered in the previous screen in accordance with an embodiment of an aspect of the present invention. Based on the information entered in screen shown in FIG. 11, the hospitality management system generates availability and pricing information. As illustrated, at the top portion of the screen 1202, information associated with hotel code 1204, room type 1206, arrival date 1208, departure date 1210, currency 1212, number of adults 1214, and number of children 1216 is indicated. At the lower portion of screen 1202, the available rates 1218 for the requested room type are displayed. The information provided for each of the available rooms comprises a contract code 1220, room type 1222, room level 1224, total cost 1226, currency (e.g., MXN: Pesos) 1228, and the cost for each night of stay 1230. Room level 1224 is a pricing level that is set by the revenue manager based on the complex real time information processing carried by the hospitality management system. Examples of such processing are illustrated in FIG. 7. If it is forecasted that rooms and/or various facilities will be in high demand, the room level may be increased, which is indicative of a price increase above a base price for a room. By activating the Add button 1232, a room of interest can be selected. Although not shown, once a room or other facility of interest is selected, a summary of the reservation details are provided on a screen in order for the agent to confirm that the requirements match that of the customer. Once this information is confirmed, the booking can be finalized by making sure the necessary payments are submitted. When the reservation is confirmed, another screen (not shown) illustrates the booking confirmation.

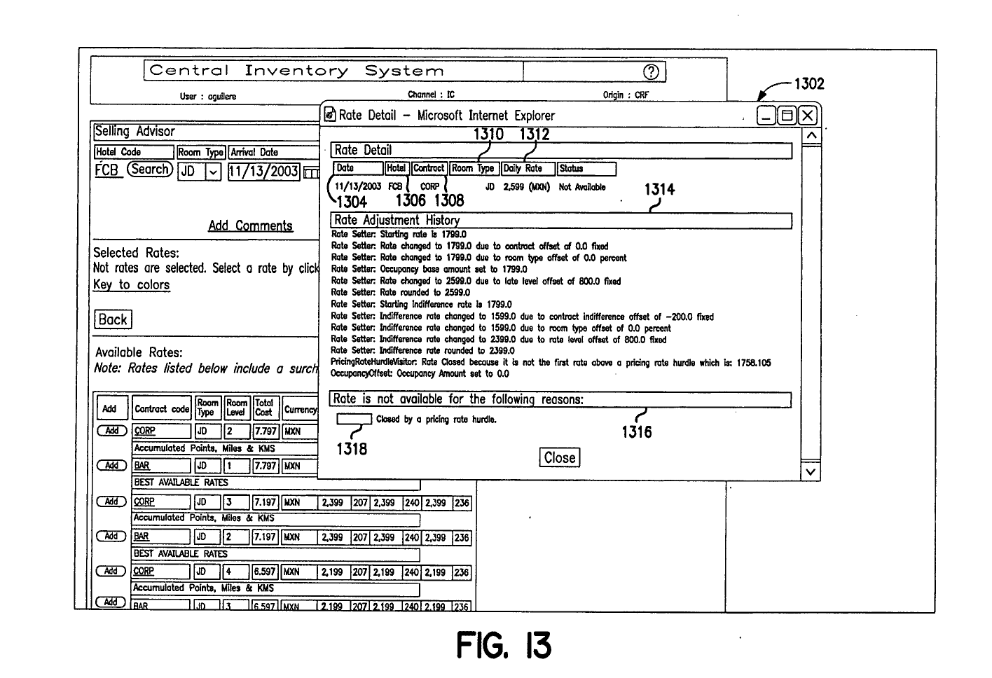

FIG. 13 shows a rate detail window 1302 that may accessed from the illustrative screen of FIG. 12, or another similar screen. As illustrated, rate details are provided based on date 1304, the hotel 1306, contract 1308, room type 1310, and daily rate 1312. The rate details are shown in the rate adjustment history section 1314, which gives an indication of how the rate has changed due to different factors. The reasons for a rate not being available is shown in section 1316. For example, the rate may not be available because it is closed by a pricing rate hurdle 1318. A pricing rate hurdle typically defines the lowest price that can be accepted by the hospitality management system for a particular booking, and is set by the revenue management system (i.e., revenue management engine and revenue management personnel).

Rate detail 1302 provides valuable information to managers or the management teams of the different business entities. Using the information available in the rate details window 1302, they can organize and determine staffing requirements, and access the reasons for particular rates not being available. This allows the management or manager to explain any pricing discrepancies perceived by customers, or it simply allows them to attend to customer relations by giving clear reasons as to why rates have gone up or down during certain periods. Also, as the management team or manager is not burdened with setting pricing rates, they can more readily attend to the running of the business entity.

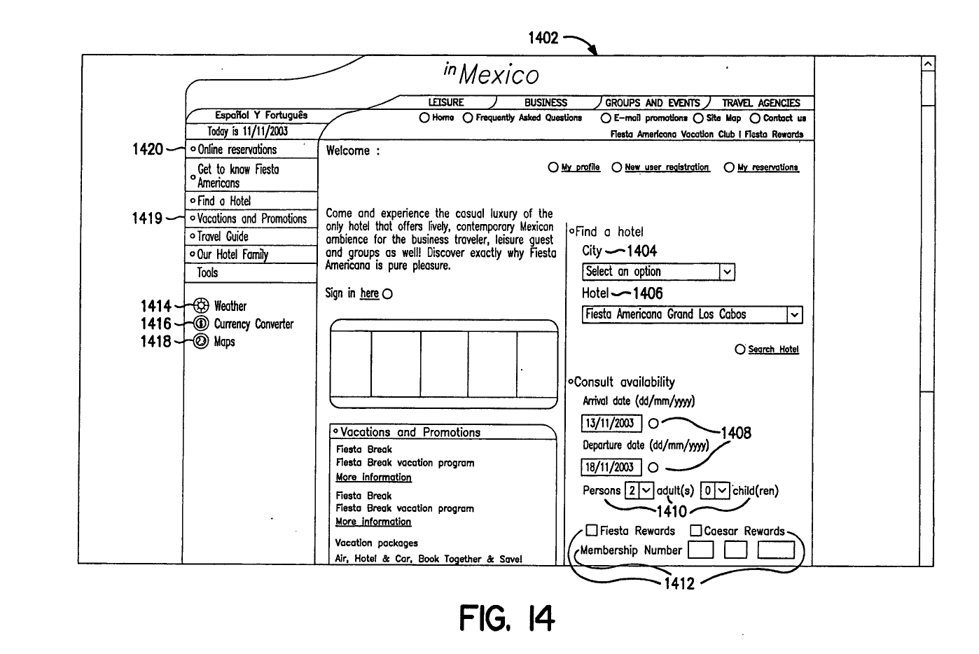

FIG. 14 illustrates an embodiment of a user interface (here, a screen-shot of an Internet Home Page) 1402 for the hospitality organization in an embodiment of an aspect of the present invention. Using the Home Page screen 1402, a customer can enter details of their proposed stay. For example, the customer may enter the required information, such as city of interest 1404, desired hotel 1406, dates of booking 1408, number of persons 1410, and membership information 1412 for points or potential discount, in order to check availability and make a booking. Other information, such as, but not limited to, whether information 1414, currency conversion 1416, maps 1418, promotions 1419, etc. are also available at the website. The customer may also directly initiate an online reservation process by selecting online reservation 1420.

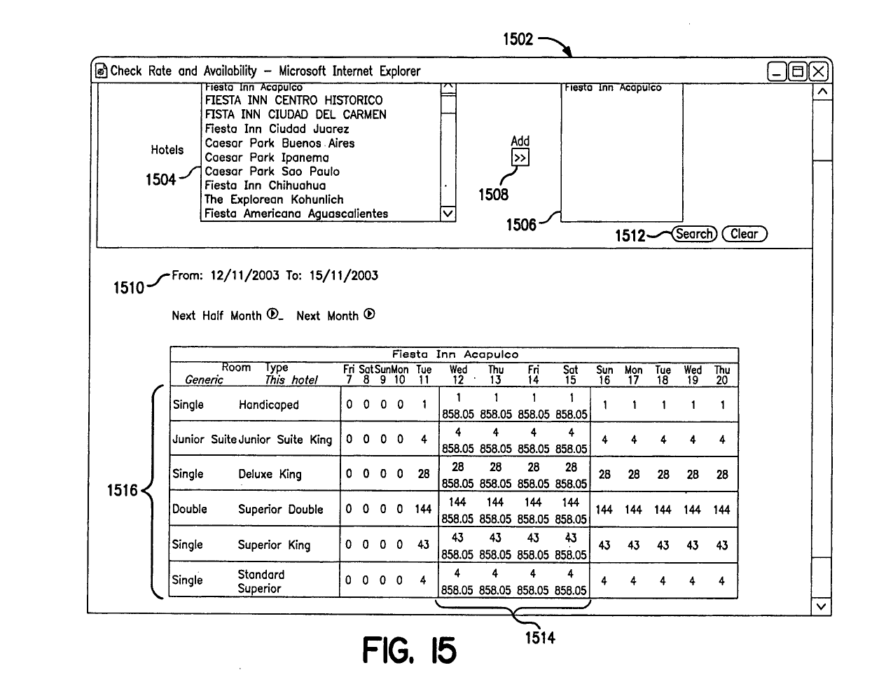

FIG. 15 illustrates an embodiment of a user interface screen-shot of a web page 1502 used by the sales force automation (SFA) system for group reservations according to an embodiment of an aspect of the present invention. As illustrated, the sales agent or sales executive may select multiple business entities from a list of hotels 1504. Each selected hotel (e.g., Fiesta Inn Acapulco) can be added to selection window 1506 by selecting the hotel and activating the add button 1508. Once the hotels of interest for a particular group and the dates of stay 1510 have been added into the selection window 1506, the search button 1512 is activated. Based on the search, which is processed by the centralized inventory system, pricing rates 1514 are provided for the different rooms 1516 associated with the selected hotels (e.g., Fiesta Inn Acapulco), based on the indicated dates of stay.

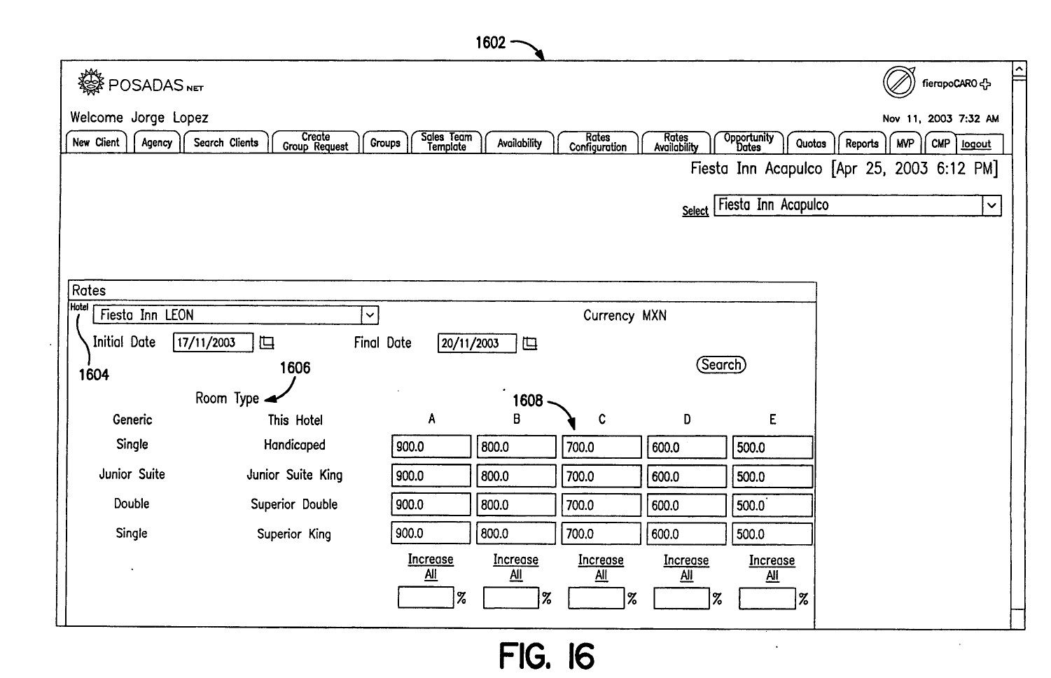

FIG. 16 illustrates an embodiment of a user interface in the form of a web page screen 1602 used by the revenue manager (person) or revenue management team according to an embodiment of an aspect of the present invention. This screen allows the revenue manager to manually override a calculated pricing rate for a particular business entity, such as, hotel 1604. For particular room types 1606, the revenue manager may manually enter a pricing rate into grid 1608.

Regardless of the different “look-and feel” of the various web pages used by the different agents (e.g., organization's sales agent, or an individual), the booking engine and the underlying business logic used for processing information entered into web pages is the same. As described in FIG. 3, the application interface (first tier) processes the different web page information, where the information is passed on to the second tier business rules logic. Hence, information entered into the Home Page (FIG. 14) or via the call reservation service (FIG. 11) are processed by the same booking engine within the application server (FIG. 3).

In addition to the embodiments of the aspects of the present invention described above, and to the extent set forth in U.S. provisional patent application No. 60/442,198, filed on Jan. 24, 2003, the contents of which are incorporated herein in their entirety, those of skill in the art will be able to arrive at a variety of other arrangements and steps which, if not explicitly described in this document, nevertheless embody the principles of the invention and fall within the scope of the appended claims. For example, the ordering of method steps is not necessarily fixed, but may be capable of being modified without departing from the scope and spirit of the present invention.

1. A method for managing a hospitality organization having geographically distributed
business entities providing one or more respective facilities, wherein arrangements with respect to use of the facilities provided by the business entities are made via one or more of a plurality of channels, the method comprising the steps of:
(a) maintaining a centralized inventory system for the business entities and the respective facilities associated with the business entities;
(b) receiving via at least one of the plurality of channels a request for a pricing proposal associated with at least one of the facilities of at least one of the business entities;
(c) in response to the request for the pricing proposal associated with the at least one facility, generating a quote based on data residing in the centralized inventory system; and
(d) transmitting the quote, via at least one of the plurality of channels, in response to the request for price information.

2. The method according to claim 1, wherein the quote is generated without using data held only by the business entity.

3. The method according to claim 1, wherein the request for a quote is received directly from a customer.

4. The method according to claim 1, wherein the hospitality organization includes sales staff members, the request for a quote is received from a sales staff member and the quote is transmitted to the sales staff member.

5. The method according to claim 1, wherein the business entity employs respective staff, the request for a quote relates to a facility of the business entity and is received from the business entity staff, and the quote is transmitted to the business entity staff.

6. The method according to claim 1, wherein the quote is generated in real time.

7. The method according to claim 1, wherein the quote is generated using real-time data residing in the centralized inventory system.

8. The method according to claim 6, wherein the quote is generated using real-time data residing in the centralized inventory system.

9. The method according to claim 1, wherein the channel via which the request for quote is received by the centralized inventory system comprises the channel via which the quote, once generated, is transmitted in response to the request.

10. The method according to claim 1, further comprising the steps of receiving an acceptance of the quote and provisionally allocating the at least one facility to which the quote relates.

11. The method according to claim 1, further comprising the step of receiving payment for the provisionally allocated at least one facility.

12. The method according to claim 11, further comprising the step of fully allocating the at least one facility.

13. The method according to claim 10, further comprising the step of updating the centralized inventory system to reflect the provisionally allocated at least one facility.

14. The method according to claim 13, wherein the centralized inventory system is updated in real time.

15. The method according to claim 11, further comprising the step of updating the centralized inventory system to reflect the receipt of payment for the provisionally allocated at least one facility.

16. The method of claim 15, wherein the centralized inventory system is updated in real time.

17. The method according to claim 12, further comprising the step of updating the centralized inventory system to reflect the full allocation of the at least one facility.

18. The method according to claim 17, wherein the centralized inventory system is updated in real time.

19. The method according to claim 1, wherein at least one of the plurality of channels comprises a global distribution system.

20. The method according to claim 1, wherein at least one of the plurality of channels comprises a property management system associated each individual one of the distributed global facilities.

21. The method according to claim 1, wherein at least one of the plurality of channels comprises a call reservation service.

22. The method according to claim 1, wherein the geographically distributed business entities comprise properties of a hotel chain.

23. The method according to claim 1, wherein at least one of the plurality of channels comprises a designated sales force, and wherein the designated sales force is associated with the hospitality organization.

24. The method according to claim 1, wherein at least one of the plurality of channels comprises an Internet site associated with the hospitality organization.

25. The method according to claim 1, wherein at least one of the plurality of channels comprises a third party Internet site for processing the request.

26. The method according to claim 1, wherein at least one of the plurality of channels comprises a customized extranet provided by the hospitality organization for a customer of the hospitality organization.

27. The method according to claim 1, wherein at least one of the plurality of channels comprises a dynamically-added channel.

28. The method according to claim 1, wherein the generated quote is independent of the channel via which the request for the quote is received.

29. A hospitality management system for providing quotes associated with facilities of geographically distributed business entities of a hospitality organization, the hospitality management system comprising:
a centralized inventory system comprising a data storage system for storage and retrieval of data associated with booking the facilities of any of the business entities; and
a central interface in communication with the centralized inventory system and the business entities and accessible by customer entities for booking at least one of the facilities of at least one of the business entities,
the centralized inventory system adapted for generating quotes based on data stored in the data storage system and associated with the facilities of the business entities.

30. The hospitality management system according to claim 29, wherein the customer entity comprises a customer.

31. The hospitality management system according to claim 29, wherein the customer entity comprises a sales entity of the hospitality organization.

32. The hospitality management system according to claim 29, wherein the data stored in and retrieved from the data storage system of the centralized inventory system comprises real-time data.

33. The hospitality management system according to claim 29, wherein the quotes generated by the centralized inventory system comprise real-time quotes.

34. The hospitality management system according to claim 29, wherein the centralized inventory system is further adapted for booking a facility of a business entity in response to acceptance of a quote by a customer entity.

35. The hospitality management system according to claim 29, wherein the central interface is adapted for receiving requests for quotes from a plurality of channels.

36. The hospitality management system according to claim 29, wherein at least a subset of the business entities communicate electronically with the hospitality management system according to procedures that differ from those of business entities not in that subset and wherein the central interface is adapted for communicating with the business entities regardless of the differing procedures.

37. The hospitality management system according to claim 36, wherein the procedures comprise data formats.

38. The hospitality management system according to claim 29, further comprising a revenue management system in communication with the centralized inventory system, the revenue management system adapted for generating quotes for facilities of the business entities according to a plurality of sources of data having potential bearing on the economic value of the facilities for which the quote is requested and under conditions imposed by the quote.

39. The hospitality management system according to claim 38, wherein the plurality of sources of data comprise real-time data from the centralized inventory system.

40. The hospitality management system according to claim 29, wherein the centralized inventory system comprises a processing system for controlling storage and retrieval of the data in the data storage system.

41. The hospitality management system according to claim 40, wherein the processing system generates the price quote based on retrieved data.

42. The hospitality management system according to claim 41, wherein the retrieved data comprises real-time data and the price quote is generated in real-time based on the retrieved real-time data.

43. The hospitality management system according to claim 29, wherein the data associated with booking of facilities of any of the business entities, stored in the data storage system, comprises real-time data.

44. The hospitality management system according to claim 29, wherein the central interface comprises a publish/subscribe system.

45. The hospitality management system according to claim 29, further comprising a business intelligence system in communication with the centralized inventory management system for accessing and processing the data associated with booking of the facilities of any of the business entities to generate customer-specific service preferences.

46. The hospitality management system according to claim 45, wherein the customer specific service preferences comprise at least one selected from the group consisting of room preferences and preferred amenities.

47. A method for operating a central inventory system for a hospitality organization having a plurality of geographically distributed business entities, the method comprising the steps of:
(a) maintaining a database associated with the central inventory system, the database comprising centrally-generated price and availability data relating to facilities of the plurality of business entities;
(b) receiving a booking request for at least one facility of the plurality of business entities;
(c) based on the booking request, retrieving from the database data relating to the facility;
(d) processing the retrieved data to generate a quote for the facility;
(e) transmitting the quote in response to the booking request;
(f) receiving a signal reflecting acceptance of the quote; and
(g) updating the database based on receipt of the signal reflecting acceptance of the quote.

48. The method for operating a central inventory system according to claim 47, wherein the database is maintained on a real-time basis.

49. The method for operating a central inventory system according to claim 47, wherein the retrieved data relating to the facility comprises price setting data.

50. The method for operating a central inventory system according to claim 49, wherein the facility is of a given type and has given characteristics, the price setting data is derived on the basis of at least one selected from the group consisting of: market analysis relating to the facility type and characteristics; strategy considerations relating to the facility type and characteristics; demand forecasting for facilities of the given type and characteristics; optimization methods for pricing of facilities of the given type; and monitoring of profitability of previously booked facilities of the given type.

51. A centralized system for managing pricing and booking of facilities of geographically distributed business entities of a hospitality organization, the centralized system comprising:
(a) a centralized inventory system for maintaining a single repository of data associated with pricing and booking of the facilities;
(b) an application server in communication with the centralized inventory system over a network, the application server being accessible over the network by the centralized inventory system for booking the facility; and
(c) a central interface in communication with the centralized inventory system, the application server and at least one external system, for supporting communications between the centralized inventory system, the application server, and the at least one external system.

52. The centralized system according to claim 51, wherein the data associated with pricing and booking of the facilities comprises real-time data.

53. The centralized system according to claim 51, wherein the external system comprises a system operated by a customer entity.

54. The centralized system according to claim 51, wherein the external system comprises a system operated by one of the plurality of business entities of the hospitality organization.

55. The centralized system according to claim 51, wherein the at least one external system comprises a property management system associated with a business entity of the hospitality organization.

56. The centralized system according to claim 51, wherein the at least one external system comprises a sales support system.

57. The centralized system according to claim 51, wherein the at least one external system comprises a revenue management system for optimizing revenues based on centralized inventory system data.

58. The centralized system according to claim 51, wherein the at least one external system comprises a global distribution system (GDS).

59. The method according to claim 51, wherein the facilities comprise properties of a hotel chain.

60. A method for managing one of a plurality of business entities of a hospitality organization, the method comprising:
(a) receiving over a network, from an inventory system centralized with respect to the plurality of business entities, data associated with booking of facilities of the business entity; and
(b) assigning resources of the business entity based on the booking data received from the centralized system.

61. The method according to claim 60, further comprising the step of accessing, over the network from the centralized inventory system, pricing data associated with the booking of facilities.

62. The method according to claim 60, further comprising the step of refraining from determining a price for the facilities of the business entity.

63. The method according to claim 60, wherein the hospitality business entity comprises a hotel.

64. The method according to claim 60, wherein the hospitality business entity comprises a cruise ship.

65. The method according to claim 61, wherein the pricing data received from the centralized inventory system are updated on a continuing basis.

66. The method according to claim 65, wherein the pricing data reflects pricing that is optimized using a revenue management system.
